(19) United States
(12) Patent Application Publication
(10) Pub. No.: US 2006/0019400 A1
(43) Pub. Date: Jan. 26, 2006

(54) Method for the determination of glucuronides in physiological samples

(75) Inventors: William R. LaCourse, Cantonsville, MD (US); Romina Kaushik, Columbia, MD (US); Ronita L. Marple, Cincinnati, OH (US)

Correspondence Address: STERNE, KESSLER, GOLDSTEIN & FOX PLLC, 1100 NEW YORK AVENUE, N.W., WASHINGTON, DC, 20005, US

(73) Assignee: University of Maryland, Baltimore, MD (US)

(21) Appl. No.: 11/186,993

(22) Filed: Jul. 22, 2005

Related U.S. Application Data
(60) Provisional application No. 60/590,805, filed on Jul. 23, 2004.

(51) Int. Cl. G01N 33/00

(52) U.S. Cl. 436/93

(57) ABSTRACT

The present invention provides methods and kits for the detection of glucuronide metabolites of various drugs, alcohols and other compounds using a combination of High Performance Liquid Chromatography coupled with Pulsed Electrochemical Detection. Detection of a drug and its glucuronide metabolite(s) has applications in interpretive forensic and clinical toxicology. The ability to estimate metabolite/drug ratios enables the assessment of route, dose and time of exposure. In instances where the parent drug is biotransformed quickly and can only found in low levels in biological fluids, the detection of metabolites allows for the identification of parent drugs. Furthermore, metabolite determination enables the differentiation between recent and chronic drug use.

### CROSS REFERENCE TO RELATED APPLICATIONS

The present application claims the benefit of U.S. Provisional Patent Application No. 60/590,805, filed Jul. 23, 2004, the disclosure of which is incorporated by reference herein in its entirety.

### BACKGROUND OF THE INVENTION

1. Field of the Invention

The present invention provides methods and kits for the detection of glucuronide metabolites of various drugs, alcohols and other compounds. Detection of a drug and its glucuronide metabolite(s) has applications in interpretive forensic and clinical toxicology. The ability to estimate metabolite/drug ratios enables the assessment of route, dose and time of exposure. In instances where the parent drug is biotransformed quickly and can only found in low levels in biological fluids, the detection of metabolites allows for the identification of parent drugs. Furthermore, metabolite determination enables the differentiation between recent and chronic drug use.

2. Background of the Invention

Metabolism is the process by which the structure of a xenobiotic is changed to facilitate its excretion from the body (Hardman, J. G., et al., Goodman & Gilman's The Pharmacological Basis Of Therapeutics, 10th ed. McGraw-Hill Inc., New York, N.Y. (2001)). Phase I metabolism comprises the enzymatic transformation of functional groups on compounds. Id. Phase II metabolism involves changing the structure of a drug or a phase I metabolite via conjugation with an endogenous substance. Id. Examples of conjugation reactions include glucuronidation and sulfate formation.

Conjugation with glucuronic acid occurs extensively in mammals and other animals (Levine, B., Principles of Forensic Toxicology, AACC Press (1999)). Glucuronidations are catalyzed by UDP-glucuronosyltransferases, located in the endoplasmic reticulum (Rashid, B. A et al., Journal of Chromatography A 797:245-250 (1998)). They catalyze the transfer of glucuronic acid from an uridinediphosphoglucuronic acid (UDPGA) cofactor to the above mentioned functional groups. Id. Compounds with carboxylic groups undergo direct conjugation with glucuronyl residues. The effect of glucuronidation is to produce an acidic compound that is more water-soluble than the parent precursor at physiological pH (Levine, B., Principles of Forensic Toxicology, AACC Press (1999)).

Ethyl Glucuronide

Alcohol is the most commonly abused substance in forensic cases. It is either found in biological samples due to alcohol consumption prior to death, or from postmortem ethanol production as part of the process of decomposition. In living individuals, analysis of gamma-glutamyltransferase (GGT), carbohydrate-deficient transferrin (CDT), 5-hydroxytryptophol (HTOL) and erythrocyte mean cellular volume (MCV) are common methods for proving chronic alcohol consumption (Seidl, S., et al., Addict Biol. 6:205 (2001)). With the exception of HTOL, all the above are indirect biomarkers of the adverse effects of impaired organs by chronic alcohol consumption that can be influenced by age, gender, genetics and a variety of substances causing abnormalities in up to 50% of the population (Wurst, F. M. et al., Alcohol 34:71 (1999)). Ethyl glucuronide (EtG) is a non-volatile, water-soluble direct metabolite of ethanol that is a highly specific and sensitive biomarker of alcohol consumption (Wurst, F. M., et al., Addict Biol. 7:427 (2002); Wurst, F. M., et al., Alcohol 20:111 (2000)). EtG bridges the gap between long-term (CDT, MCV & GGT) and very short-term (ethanol & HTOL) biomarkers (Wurst, F. M., et al., Addiction 98:51 (2003)). Furthermore, EtG can be a marker of alcohol consumed at low levels, and, unlike HTOL or ethanol, it can be detected for an extended period (up to 80 h) after the complete elimination of alcohol from the body (Wurst, F. M., et al., Alcohol 20:111 (2000)).

EtG was first isolated in 1952 by Kamil et al. from rabbit's urine (Schmitt, G., et al., Journal of Forensic Sciences. 42(6):1099-1102 (1997)). In 1967, Jaakonmaki et al. detected the metabolite in human urine (Schmitt, G., et al., Journal of Forensic Sciences. 42(6):1099-1102 (1997)). Urine samples taken before ethanol consumption or from teetotalers lack EtG, suggesting it is only formed after consumption of alcohol (Zimmer, H., et al., Journal of Analytical Toxicology. 26 (1):11-16 (2002)).

Conjugation of ethanol with glucuronic acid occurs in the endoplasmic reticulum of liver cells and to a lesser degree in cells of the intestinal mucosa and lung (Seidl, S., et al., Addiction Biology 6:205-212 (2001)). Glucuronidation of ethanol requires activated glucuronic acid and the presence of UDP-glucuronyl transferase (Stephanson, N., et al., Therapeutic Drug Monitoring. 24:645-651 (2002)). Past drinking studies have found a phase delay in the EtG concentration curve following alcohol intake in comparison to the ethanol curve, but that EtG is detected for a much longer period than ethanol (Wurst, F. et al., Addiction Biology 7:427-434 (2002)).

Various methods used to detect EtG include gas chromatography (GC) coupled with mass spectrometry (MS), and liquid chromatography (LC) coupled with MS (Wurst et al., Alcohol and Alcoholism. 34 (1):71-77 (1999)), are complicated and expensive. Zimmer et al. have developed an enzyme-linked immunosorbent assay (ELISA) to detect EtG (Zimmer, H., et al., Journal of Analytical Toxicology. 26 (1):11-16 (2002)). However, this method is hampered by false positive and false negative readings. For widespread use of EtG as a marker of alcohol consumption, simple, less expensive methods that reduce the number of incorrect readings are needed. The present invention fulfills these needs, by providing methods and kits for EtG detection based on High Performance Liquid Chromatography coupled with Pulsed Electrochemical Detection.

### BRIEF SUMMARY OF THE INVENTION

In one embodiment, the present invention provides methods for detecting one or more glucuronide metabolites in a liquid sample, comprising: adding an organic solvent to the liquid sample to form a mixture; passing the mixture through one or more analytical chromatographic columns, thereby separating the one or more glucuronide metabolites and producing an eluate; adding NaOH to the eluate; and detecting one or more glucuronide components of the separated glucuronide metabolites with an electrochemical detector. In other embodiments, the methods can further comprise passing the mixture in through one or more pre-concentration chromatographic columns, thereby retaining the glucuronide metabolites on the pre-concentration chromatographic columns and concentrating the glucuronide metabolites; and delivering a solvent to the pre-concentration chromatographic columns, thereby eluting the glucuronide metabolites from the pre-concentration chromatographic columns to form a mixture to be passed through the analytical column.

The methods can be used to detect any glucuronide metabolite, for example, glucuronide components produced by glucuronidation of an alcohol, morphine, cannabinoid, an androgen, acetaminophen, codeine, buprenorphine or tramadol. The methods are also useful to detect individual glucuronides in a mixture of metabolites. Physiologic samples, such as urine can be analyzed using the methods disclosed herein. In certain embodiments, the methods are useful for the detection of alcohol in urine.

The present invention also provides glucuronide analysis kits comprising: one or more chromatographic columns; one or more organic solvents; one or more glucuronide standards; and NaOH.

In another embodiment, the present invention provides methods for determining the prior consumption of a drug or alcohol by an animal, comprising: obtaining a physiologic liquid sample from the animal comprising one or more glucuronide metabolites of the drug or alcohol; adding an organic solvent to the liquid sample to form a mixture; passing the mixture through one or more analytical chromatographic columns, thereby separating one or more glucuronide metabolites and producing an eluate; adding NaOH to the eluate; detecting one or more glucuronide components of the separated glucuronide metabolites with an electrochemical detector; and correlating the one or more glucuronides detected with one or more drugs or alcohols consumed by the animal.

### BRIEF DESCRIPTION OF THE FIGURES

The foregoing and other features and advantages of the invention will be apparent from the more particular description of the invention, as illustrated in the accompanying drawings. The drawings are not to scale.

FIG. 1A shows a glucuronide detection apparatus in accordance with one embodiment of the present invention.

FIG. 1B shows a flowchart of a method for detecting glucuronides in accordance with one embodiment of the present invention.

FIG. 2 shows a plot of retention factor (k′) versus percent acetonitrile.

FIG. 3 shows background corrected PV responses as a function of detection potential for ( [symbol] ) MetG, 40 μg/mL, at a Au RDE in ( [symbol] ) 0.20 M NaOH. Conditions: 900 rpm rotation speed.

FIG. 4 shows PV responses of MetG in various amounts of 200 mM NaOH.

FIG. 5 shows PV responses of MetG in various amounts of mobile phase/NaOH.

FIG. 6 shows PV response of methyl glucuronide generated with 200 mM NaOH.

FIG. 7 shows PV response of methyl glucuronide with the addition of 2% acetonitrile.

FIG. 8 shows PV response of MetG in 5% methanol.

FIG. 9 shows an exemplary PED waveform for detection of glucuronides.

FIG. 10 shows a PED chromatogram of MetG and EtG at 10 μg/mL. Conditions: column, Denali C18 monomeric 100A Vydac column (4.6 mm×250 mm) with guard; mobile phase: 1% acetic acid/2% acetonitrile (98:2, v/v); flow-rate, 1.0 ml/min; post-column reagent, 600 mM NaOH at 0.5 mL/min, and optimized PED waveform (Table I).

FIG. 11 shows a representative chromatogram of 100 ug/mL EtG water standard.

FIG. 12 shows the calibration curve for water spiked (unextracted) with EtG at different concentrations.

FIG. 13 shows a calibration curve of EtG spiked into urine.

FIG. 14 shows recovery plots for EtG solutions assayed (◯) before and (□) after SPE.

FIG. 15 shows a chromatograph of a postmortem case sample.

FIG. 16 is a chromatogram of postmortem case #3594 with MetG spiked into the sample serving as the internal standard.

FIG. 17 shows 3 postmortem case chromatograms overlapped with different levels of EtG.

FIGS. 18A-18C show correlation between EtG levels (μg/mL) and (A) vitreous humor, (B) blood, and (C) urine alcohol concentration (μg/mL). The dotted lines represent the 95% confidence interval for the correlation plot. EtG levels were determined using HPLC-PED preceded by SPE. VHAC, BAC, and UAC levels were determined by GC Headspace analysis at the Office of the chief medical examiner, Baltimore, Md.

FIG. 19 shows standard calibration curves of the morphine glucuronides.

FIG. 20 is a representative chromatograph of the morphine glucuronide M3G.

FIG. 21 is a representative chromatograph of the morphine glucuronide M6G.

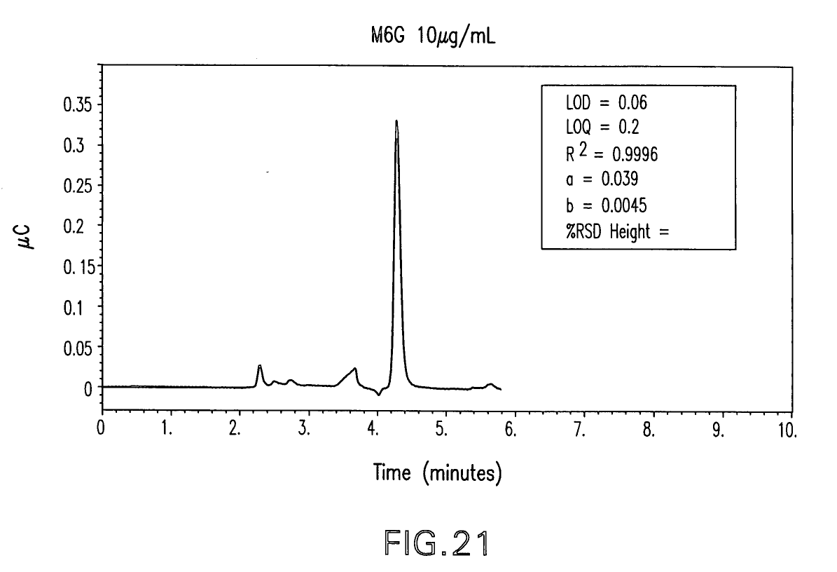

FIG. 22 is a representative chromatograph of acetaminophen glucuronide.

FIG. 23 is a representative chromatograph showing separation of a mixture of seven glucuronides by reversed-phase liquid chromatography with pulsed electrochemical detection. Peaks: (1) methyl glucuronide; (2) ethyl glucuronide; (3) morphine-3-glucuronide; (4) acetaminophen glucuronide; (5) morphine-6-glucuronide; (6) codeine-6-glucuronide; and (7) phenyl glucuronide.

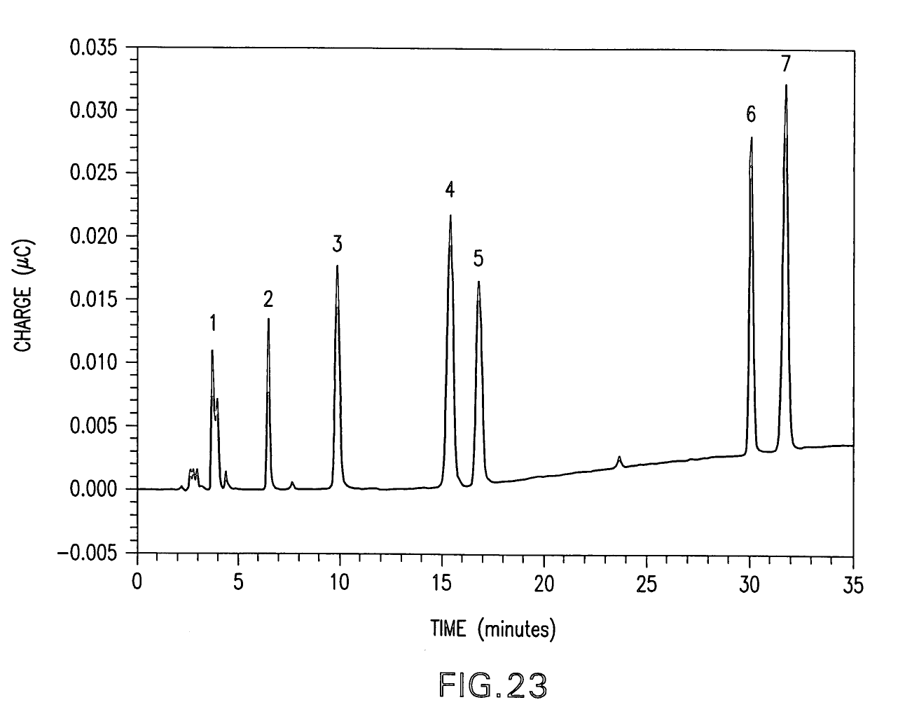

### DETAILED DESCRIPTION OF THE INVENTION

Suitable embodiments of the present invention are now described with reference to the figures, where like reference numbers indicate identical or functionally similar elements. While specific configurations and arrangements are discussed, it should be understood that this is done for illustrative purposes only. A person skilled in the relevant art will recognize that other configurations and arrangements can be used without departing from the spirit and scope of the invention.

Matrix Components and Characteristics

Although glucuronides are detectable in the blood and bile, they are generally found in the highest concentrations in the urine. Urine is a complex matrix that is generally free of protein and lipids, and as a result, analytes can be easily extracted with an organic solvent (Chamberlain, J., The analysis of drugs in biological fluids, CRC Press, Inc., Boca Raton, Fla. (1995)). Solid phase extraction (SPE) allows for concentration of a glucuronide metabolite sample and preparation for use in High Performance Liquid Chromatography (HPLC).

High Performance Liquid Chromatography (HPLC)

HPLC is a separation technique where the stationary phase is a solid and the mobile phase is a liquid. HPLC is generally divided into normal phase and reverse phase. Normal phase HPLC uses a non-polar mobile phase and a polar stationary phase. Reverse phase HPLC uses a polar mobile phase and a non-polar stationary phase. In reverse phase HPLC, C18 stationary phases are the most common. Compounds that are more nonpolar are better retained by the reversed-phase surface (Meyer, V. R., Practical High-Performance Liquid Chromatography 3rd Ed. John Wiley & Sons, New York, N.Y. (2000)). The partition of the sample components between the two phases will depend on their respective solubility characteristics. Less hydrophobic components will end up primarily in the hydrophilic phase while more hydrophobic substances will be found in the lipophilic phase. A high concentration of the organic solvent will increase the extractive power for hydrophobic compounds. Depending on the extractive power of the eluant, a greater or lesser part of the sample component will be retained reversibly by the stationary phase. The larger the fraction retained in the stationary phase, the slower the sample component will move through the column. Hydrophilic compounds will always move faster than hydrophobic ones, since the mobile phase is always more hydrophilic than the stationary phase.

Pulsed Electrochemical Detection

Pulsed Electrochemical Detection (PED) exploits the electrocatalytic activity of noble metal electrode surfaces to oxidize various polar functional groups. In PED, multi-step potential-time waveforms at Gold (Au) and Platinum (Pt) electrodes realize amperometric/coulometric detection while maintaining uniform and reproducible electrode activity. The response mechanisms in PED are dominated by the surface properties of the electrodes, and, as a consequence, members of each chemical class of compounds produce virtually identical voltammetric responses. Thus, PED requires a priori separation of complex mixtures via chromatographic or electrophoretic means.

Pulsed electrochemical detection (PED) allows for the direct detection of glucuronides following reversed-phase HPLC. Post-column chemical derivatization produces electroactive compounds that are charged to allow detection. The addition of post-column sodium hydroxide (NaOH) creates a basic environment rendering glucuronides charged for PED. The addition of NaOH can also occur pre-column in other embodiments, so long as it is present prior to PED analysis. PED employs alternated positive and negative potential pulses to clean and reactivate noble metal electrodes that have become fouled by adsorbed carbonaceous materials.

Detection of Glucuronides

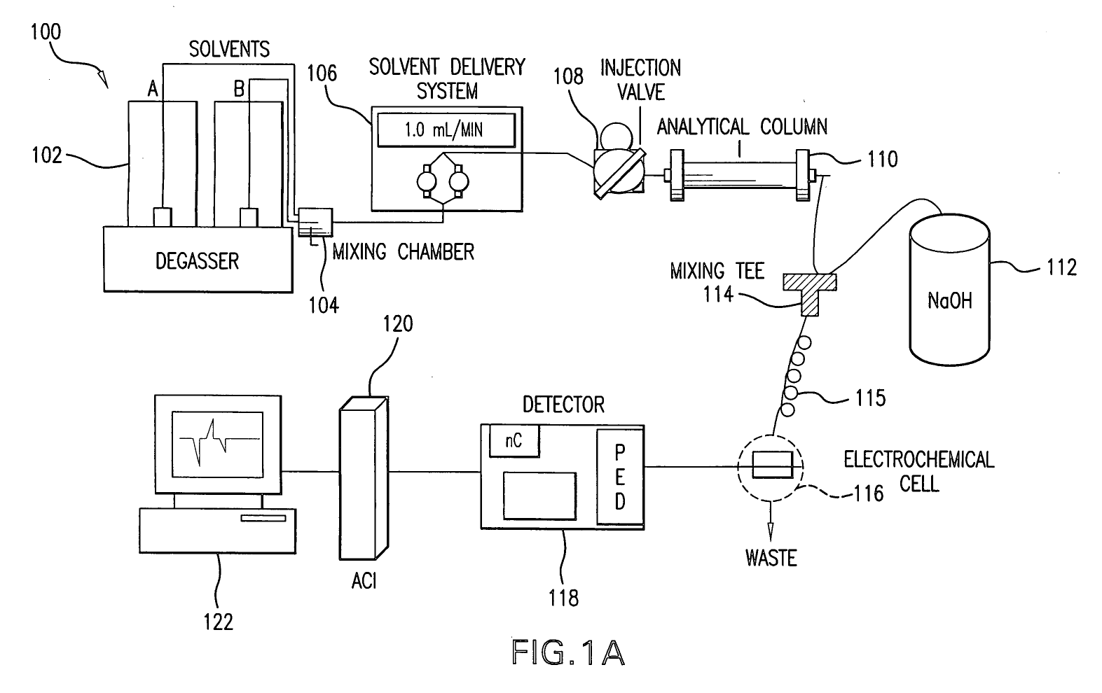

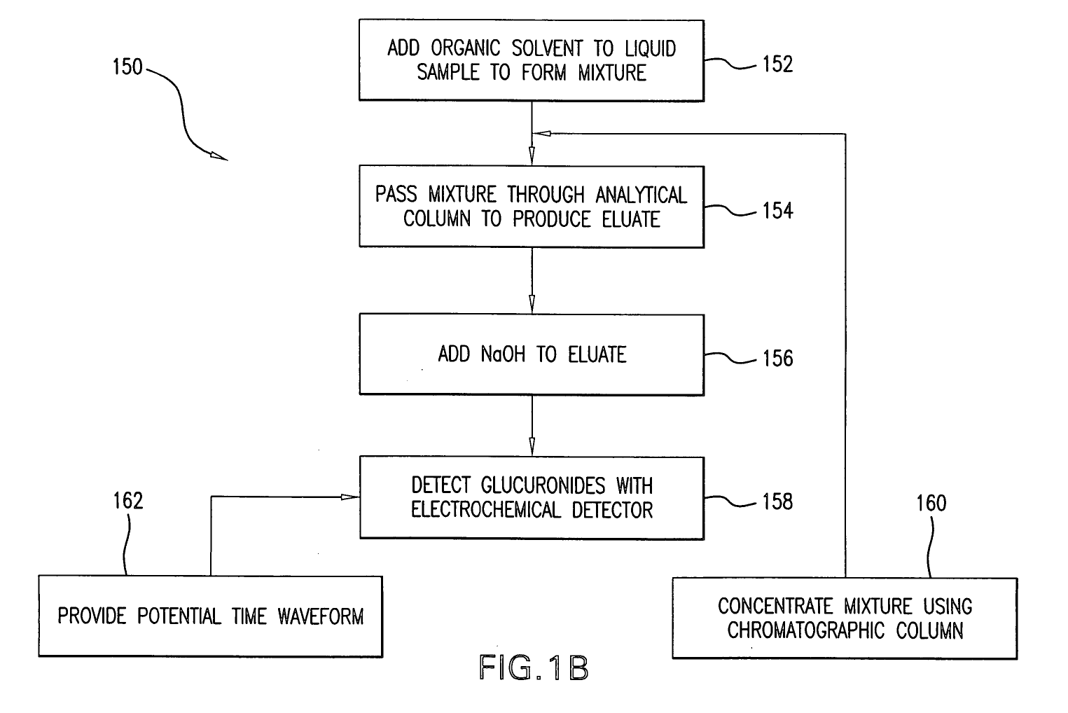

FIG. 1A shows a diagram of a generic analytical apparatus 100 used to practice the various glucuronide detection methods disclosed herein. A ternary gradient solvent delivery pump 106 (Model 8800; Spectra Physics, Mountain View, Calif.) equipped with mixing chamber 104 and on-line degasser unit 102 (Model KT-35M; Shodex, Clear Brook, Va.) were used to provide solvent delivery to analytical column 110. In certain embodiments, isocratic separations are performed using a 1% acetic acid/water:acetonitrile (98:2, v/v) mobile phase set to a flow rate of 1.0 mL [symbol] min-1. Injection valve 108 (Model 7010; Rheodyne, Rohnert Park, Calif.) is fitted with a 50 μL injection loop. Separations can be performed using any suitable analytical column, selected based upon the glucuronide(s) of interest. For example, a reversed-phase (C18; 250×4.6 mm, 5 μm, 100 Å) Denali column (Vydac, Columbia, Md.) with guard column (C18; 7.5×4.6 mm, 5 μm; Vydac) can be used, or a bonded-phase silica column comprising a C18 functional group bonded to its surface using a sulfonamide group coupled to an ether linkage (DIONEX® PA column, DIONEX®, Sunnyvale, Calif.) can be used. Post-column addition of NaOH 112 (for example, 600 mM NaOH), necessary to promote PED-activity, was accomplished by a reagent delivery module (RDM, Dionex, Sunnyvale, Calif.). The post-column apparatus is placed between analytical column 110 and electrochemical detector 118. NaOH reagent is added (e.g., at a flow rate of 0.6 mL [symbol] min-1) to the eluant from analytical column 110 via mixing-tee 114, and is then passed to a 1.0 mL, knitted, Teflon® tubing reaction coil 115 to allow sufficient mixing and to achieve a final solution of approximately 0.2M NaOH. PED was accomplished using a pulsed electrochemical detector (Dionex) 118 employing a modified quad-potential waveform. Electrochemical cell 116 consists of an Au working electrode (3.0 mm diameter), a Ag/AgCl reference electrode (model 42442; Dionex), and a stainless steel body serving as the auxiliary electrode. Electrochemical cell 116 can also be a modified model ED40 electrochemical cell (Dionex) with a 1.0 mm diameter Au electrode. Data acquisition and instrument control was accomplished with an advanced computer interface 120 (Dionex) using Dionex PeakNet software, version 5.21, on a Gateway Pentium III computer 122.

FIG. 1B, with reference to the apparatus shown in FIG. 1A, shows a flowchart 150 of a method for detecting one or more glucuronide metabolites in a liquid sample in accordance with one embodiment of the present invention, comprising: in step 152 of FIG. 1B, adding an organic solvent to the liquid sample to form a mixture, (e.g., using degasser 102, mixing chamber 104 and solvent delivery system 106). In step 154 of FIG. 1B, the mixture is then passed through one or more analytical chromatographic columns 110 (e.g., using injection valve 108), thereby separating the one or more glucuronide metabolites and producing an eluate. In step 156 of FIG. 1B, NaOH 112 is added to the eluate (e.g., in a mixing tee 114 and tubing reaction coil 115). In step 158 of FIG. 1B, one or more glucuronide components of the separated glucuronide metabolites are then detected with an electrochemical detector 118 (e.g., employing an electrochemical cell 116).

Any, and all, glucuronide metabolites present in a liquid sample can be detected via the methods disclosed herein using a combination of column separation and pulsed electrochemical detection. The disclosed detection methods allow for the detection of the sugar, i.e. the glucuronide, of the various glucuronide metabolites. This unique system allows for the separation and then detection of any number of glucuronide metabolites that may be present, thereby giving information on the complete makeup of a liquid sample and thus the complete metabolic history of a patient.

While any glucuronide metabolite of a drug, alcohol or other compound can be detected using the methods disclosed here, suitable classes and examples of glucuronides include, but are not limited to, alcohol glucuronides (e.g., methyl, ethyl, propyl and butyl glucuronides), morphine glucuronides (e.g., morphine-3- and morphine-6-glucuronides), cannabinoid glucuronides, androgen glucuronides (e.g., testosterone, epitestosterone, adrosterone, etiocholanalone, 11-ketoandrosterone, 11-ketoetiocholanolone, 11β-hydroxyandrosterone, 11β-hydroxyetiocholanolone, dehydro-epiandrosterone-3-glucuronide, dihydrotestosterone and testosterone-17-glucuronide glucuronides), acetaminophen glucuronides, opiate glucuronides, codeine glucuronides (e.g., codeine-6-glucuronide), buprenorphine glucuronides, tramadol glucuronides, and tetrahydrocannabinol glucuronides (e.g., THC-COOH glucuronides).

While any liquid sample can be analyzed for the presence (or absence) of various glucuronides, suitably the liquid sample will be a physiologic sample from a human or animal patient, such as blood or urine. As urine is generally easier to obtain and prepare for analysis, it is generally the preferred physiologic sample type. As such, the present invention therefore provides a non-invasive method for determining the presence (or absence) of various glucuronides.

In order to provide a more concentrated and purified glucuronide sample to be introduced to the analytical column, solid phase extraction (SPE) can be used prior to separation and subsequent detection. As such, the mixture produced in step 152 of FIG. 1B can be passed through one or more pre-concentration chromatographic columns, thereby retaining the one or more glucuronide metabolites on the pre-concentration chromatographic columns and concentrating the glucuronide metabolites, as in 160 of FIG. 1B. Then, by delivering a solvent to the pre-concentration chromatographic columns, the glucuronide metabolites are eluted from the pre-concentration chromatographic columns to form a mixture that can then be passed through analytical column 110, as in step 154 of FIG. 1B.

As noted above, pulsed electrochemical detection requires the use of a potential waveform to allow for cleaning and reactivating the noble metal electrodes that have become fouled by adsorbed carbonaceous materials. As shown in 162 of FIG. 1B, the methods disclosed herein can further comprise providing a potential time waveform to an electrode of the electrochemical detector 118 in order to allow for glucuronide detection. The particular waveform used depends on the type of glucuronide(s) detection that is desired. The waveform discussed below in the Examples was specifically designed for the analysis of ethyl glucuronide. Those skilled in the art will be able to envision other waveforms necessary to detect other glucuronide metabolites.

In one embodiment, the present invention provides methods for the detection of glucuronide metabolites of alcohol, including ethyl glucuronide (EtG), in liquid samples, including physiologic samples such as blood, and more suitably urine. Ethyl glucuronide is not produced as a result of postmortem decomposition, as the enzymes that produce it stop functioning immediately after death. Therefore, this metabolite is indicative of alcohol consumption, produced by liver enzymes as part of the excretion process (Wurst, F. et al., Alcohol 20:111-116). The methods disclosed herein employ pulsed electrochemical detection (PED), as the detection technique. To separate EtG from the urine matrix, a solid phase extraction technique has been used.

In another embodiment, the present invention provides methods for detecting one or more alcohol glucuronides of a liquid sample, comprising: adding an organic solvent to the liquid sample to form a mixture; passing the mixture through one or more analytical chromatographic columns, thereby separating the one or more alcohol glucuronides; adding NaOH to the separated one or more alcohol glucuronide; and detecting one or more glucuronide components of the separated alcohol glucuronides in an electrochemical detector.

As with the general class of glucuronide metabolites, solid phase extraction to concentrate and purify the alcohol glucuronides present in the liquid sample prior to separation on the analytical column can also be used. Suitable alcohol glucuronides that can be detected using the methods disclosed herein include, but are not limited to, methyl glucuronide, ethyl glucuronide, butyl glucuronide and propyl glucuronide.

While any analytical chromatographic column(s) and solvent systems can be used in the practice of the methods disclosed herein, for separation of alcohol glucuronides, including ethyl glucuronide (EtG) a Denali reversed phase analytical column, or a bonded-phase silica column comprising a C18 functional group bonded to its surface using a sulfonamide group coupled to an ether linkage (DIONEX® PA column, DIONEX®, Sunnyvale, Calif.) and t-butyl alcohol are suitably used.

The present invention also provides for glucuronide analysis kits comprising: one or more chromatographic columns; one or more organic solvents; one or more glucuronide standards; and NaOH. Suitable chromatographic columns and solvents include those disclosed herein, such as reversed phase Denali columns and t-butyl alcohol.

In order to properly detect each specific glucuronide metabolite in a liquid sample, an individual standard designed for each metabolite “class” is required. For example, methyl glucuronide can be used as a standard when analyzing for various other alcohol glucuronides. Similarly, related standards for other glucuronides can also be part of the kits disclosed herein.

In another embodiment, the present invention provides methods for determining the prior consumption of a drug or alcohol by an animal, comprising: obtaining a physiologic liquid sample from the animal comprising one or more glucuronide metabolites of the drug or alcohol; adding an organic solvent to the liquid sample to form a mixture; passing the mixture through one or more analytical chromatographic columns, thereby separating one or more glucuronide metabolites and producing an eluate; adding NaOH to the eluate; detecting one or more glucuronide components of the separated glucuronide metabolites with an electrochemical detector; and correlating the one or more glucuronides detected with one or more drugs or alcohols consumed by the animal. As used herein, the term animal is meant to encompass animals of any species, including, but not limited to, mice, rats, rabbits, horses, hamsters, guinea pigs, pigs, micro-pigs, goats, sheep, cows, non-human primates (e.g., baboons, monkeys, and chimpanzees), as well as humans. The methods of the present invention are suitably used for the determination of prior drug or alcohol consumption by a human.

It will be readily apparent to one of ordinary skill in the relevant arts that other suitable modifications and adaptations to the methods and applications described herein may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Ethyl Glucuronide Detection

Materials And Methods

Reagents and Solutions

Ethyl glucuronide (EtG) standard was obtained from Medichem, (Steinenbronn, Germany). Methyl glucuronide (MetG) was obtained from Sigma Chemical Company (St.Louis, Mo.). All working solutions were made with 3× filtered Reverse Osmosis deionized water (U.S. Filter/IONPURE, Lowell, Mass.). Post-column NaOH, 600 mM was prepared from pure NaOH purchased from VWR Scientific Products Corporation (Baltimore, Md.). Postmortem urine was obtained from the Office of the Chief Medical Examiner, State of Maryland.

Instrumentation

The HPLC and PED system used in the analysis are described above and diagramed in FIG. 1A.

Pulsed voltammetry (PV) experiments were carried out on a model AFRDE4 Bi-Potentiostat from Pine Instrument Company (Grove City, Pa.). PV waveforms were generated with ASYST scientific software (Asyst Software Technologies, Rochester, N.Y.) on a 286/16 MHz IBM compatible computer interfaced via a DAS-20 AD/DA expansion board (Keithley Data Acquisition, Taunton, Mass.). All voltammetric experiments were performed using a Au rotating disk electrode (3 mm diameter). The auxiliary electrode was a platinum wire, and the reference electrode was an Ag/AgCl electrode (Model MR-5275; Bioanalytical Systems, West Lafayette, Ind.). The electrochemical cell (˜125 mL) was constructed of Pyrex glass with two side arms separated from the cell body with fine glass frits and filled with 0.2 M NaOH.

Sample Extraction

All urine samples (1.0 mL) were pretreated with 100 μL of 3 M HCl and 3 mL acetonitrile. A 100 μL aliquot of 4000 μg/mL MetG solution was spiked into all pretreated samples as an internal standard. The entire sample was loaded onto a SPE cartridge (Sep-Pak; Waters, Milford, Mass.) containing 500 mg (3 cc) sorbent bed of aminopropyl stationary phase at a rate of ˜0.5 mL·min-1. Solid Phase Extraction was performed on a Speed-Mate 10 vacuum manifold apparatus (Applied Separations; Allentown, Pa.). Aminopropyl cartridges were conditioned by consecutively loading and removing 3 mL methanol, 3 mL distilled water and 3 mL acetonitrile prior to sample loading. The loaded sample was subsequently pulled through the device by application of a vacuum, and the cartridge was washed with 3 mL of acetonitrile. A vacuum (˜15 mm Hg) was applied for approximately 10 minutes to remove any aqueous residue. Finally, the glucuronides were eluted twice with 2 mL aliquots of 2% ammonia in methanol into a 12×75 mm disposable culture tube, dried down in a Speed Vac (Model SC110; Savant, Albertville, Mich.), and reconstituted with 1.0 mL of water. The reconstituted solution was diluted further to bring EtG within the linear range of quantitation.

Results and Discussion

Solid Phase Extraction Theory and Development

Urine samples were pretreated in order to protonate and render the glucuronide in its neutral state. A methyl glucuronide solution for use as a standard was spiked into all pretreated samples at a final concentration of 100 ppm. For conditioning, a solvent was passed through the sorbent to wet the packing material and functional groups of the sorbent. Air present in the column was removed and empty spaces were filled by the solvent. A suitable conditioning solvent is methanol, followed by water activating the column. The volume of the sample loaded (dependent on the capacity of the SPE cartridge) is pulled through with a vacuum pump. Normal phase sorbents have a stationary phase that is more polar than the sample applied to the SPE cartridge. In its neutral state, the glucuronide hydrogen bonds to the aminopropyl groups on the cartridge thereby concentrating the analyte on the sorbent. In addition, desired components are retained on the sorbent, while others pass through, giving partial purification of the analyte. Next, the sorbent is rinsed to remove interfering matrix components but retain the analyte of interest. Acetonitrile was used as a washing solution. If the sample is pretreated with an organic solvent, it can also serve as the rinse solvent. A constant vacuum was applied to each cartridge after the wash for 10 minutes to remove any aqueous residue to optimize recovery. Finally, elution with an appropriate solvent disrupts the analyte-sorbent interaction and enables collection of the analyte.

Removal of other substances adsorbed on the column by the eluting solvent should be minimal. The sample is eluted from the sorbent using polar solvents that disrupt the hydrogen bonding between functional groups of the analyte and the sorbent surface. Methanol is one example base solvent for elution. In one embodiment, 2% ammonia with methanol was used as the elution solvent. The smallest volume of solvent should generally be used so as to concentrate the analytes. A two step elution was performed resulting in improved recovery of the analyte. The eluate was then dried down and reconstituted in water for HPLC analysis.

Ethyl glucuronide standard was used for the development of a solid phase extraction protocol. Initially, C18 cartridges with a capacity to hold 3 mL sample volume were tested for EtG extraction, but no peak was resolved using this column. As a result, more polar aminopropyl cartridges were evaluated as a substitute for the C18 cartridges. The extraction was initially done with isopropanol as the wash solvent. This yielded a very dirty extraction but the EtG peak was visible in the chromatograms. Next, n-hexane was used as the wash solvent. However, dirty extracts were still generated. Acetonitrile was ultimately selected as the wash solvent for subsequent extractions as it gave the cleanest extraction. One step, 2 mL elution with 2% ammonia in water, 2% ammonia in methanol, and two step elutions with 2 mL elution solvent per step were evaluated, with the two step elution resulting in higher recovery. A 3 cc (500 mg sorbent) was used for all extractions.

Chromatography

Determination of Mobile Phase Composition

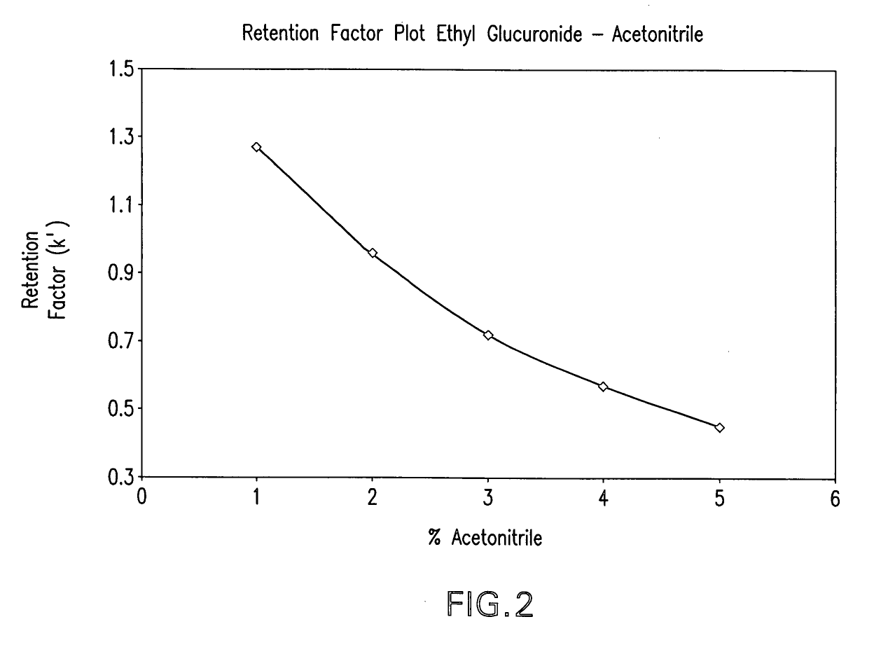

Using a mixture of water and acetonitrile (ACN) was selected as the mobile phase and a plot of retention factor (k′) plot versus percent acetonitrile was generated (FIG. 2). As the percentage of organic was increased, the retention time of the analyte decreased. These plots are useful when comparing the retention times of different compounds to determine the % organic at which the compounds separate from each other in the shortest retention time. Furthermore, different columns can be compared to determine the best column for separation with shortest retention time. Various percentages of ACN were tested and 2% ACN was selected for the mobile phase because the analyte separated from the interfering components of the urine. 1% acetic acid in combination with 2% ACN/water was used as the mobile phase as it is not electroactive. This selection resulted in completely resolved peaks and a stable baseline.

Determinations of Conditions of Post-Column Delivery and PED Waveform

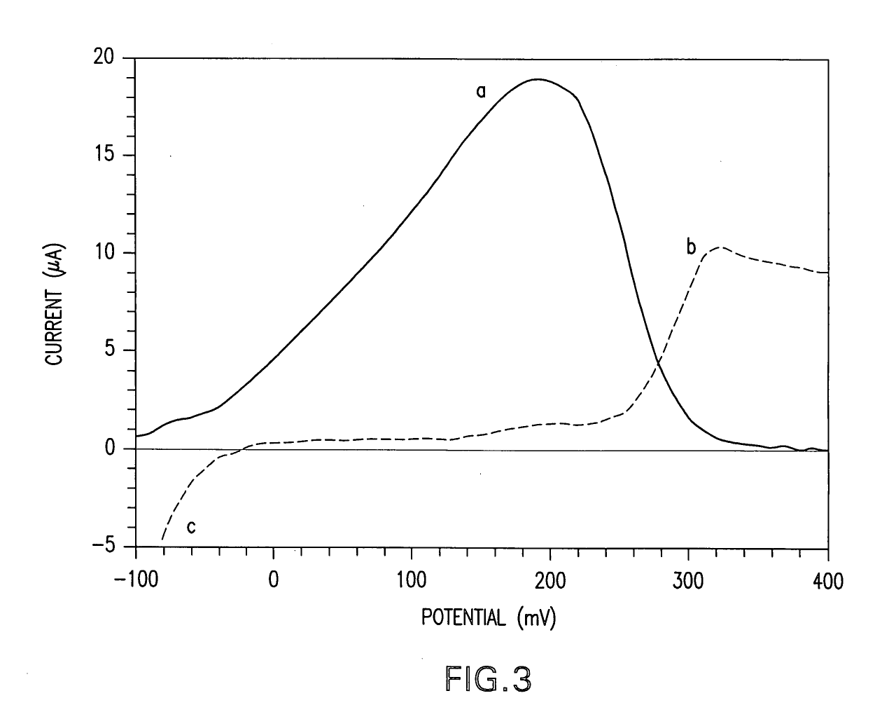

FIG. 3 shows the background-corrected current to potential response of ( [symbol] ) MetG at a Au RDE ( [symbol] ) in 0.2M NaOH. MetG shows an anodic wave beginning at approximately −100 mV due to the electrocatalytic oxidation of the hydroxyl groups. The signal is attenuated beyond approximately +250 mV due to the formation of surface oxide, which is clearly shown by the anodic wave (wave a) commencing at approximately +250 mV on the voltammogram of the supporting electrolyte, inhibiting detection of carbohydrates. If dissolved O₂ is present in the solution, its reduction is observed as a cathodic wave commencing at approximately −20 mV. The potential region between dissolved O₂ reduction (wave c) and the onset of oxide formation (wave b) is typically denoted as the “oxide-free” window, where carbohydrate detection can be monitored with the least background interference. The optimized detection potential was determined to be +200 mV based on a maximization of signal-to-noise for MetG and, hence, virtually all glucuronides.

PV studies were conducted to determine the optimal waveform parameters as well as the appropriate NaOH concentration for detection purposes. For these studies methyl glucuronide was used. A series of PV experiments to determine the effect of acetonitrile on detection showed that detection of glucuronic acid is suppressed by presence of acetonitrile.

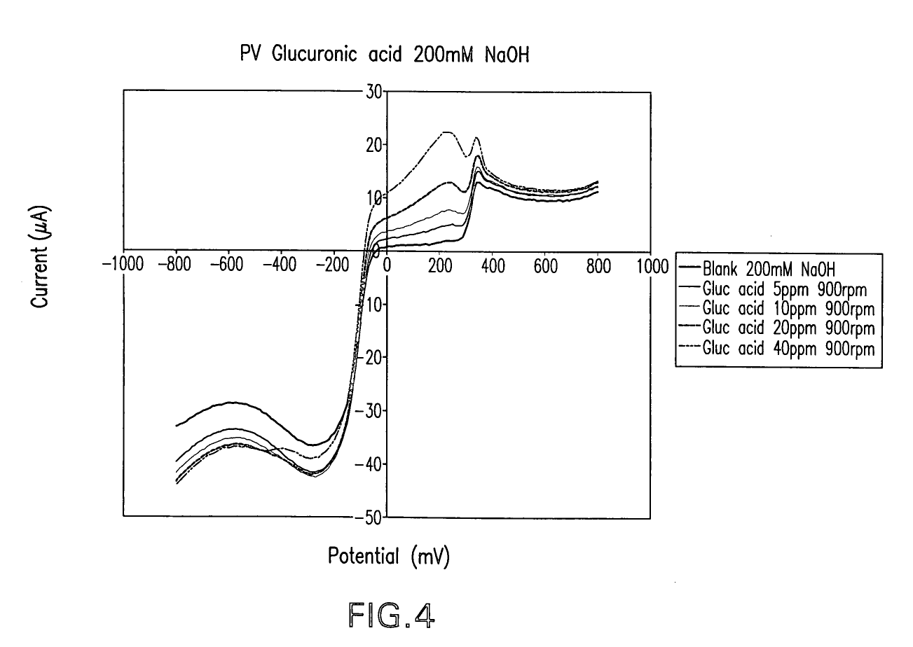

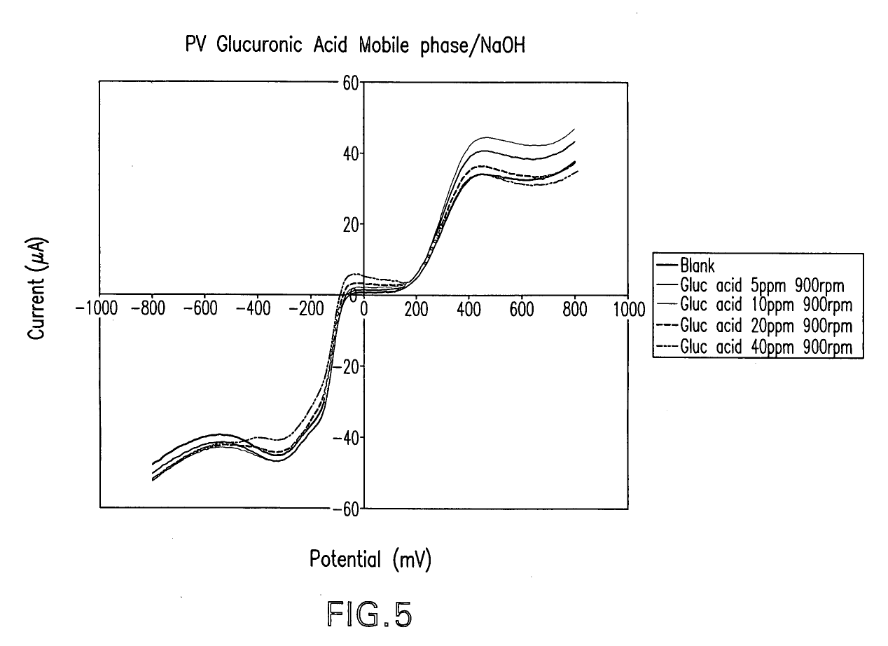

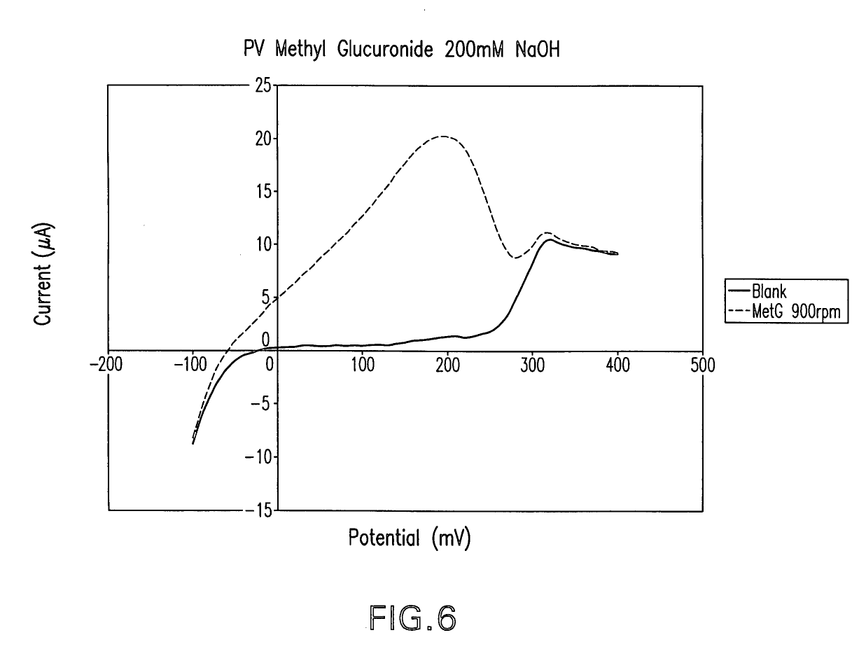

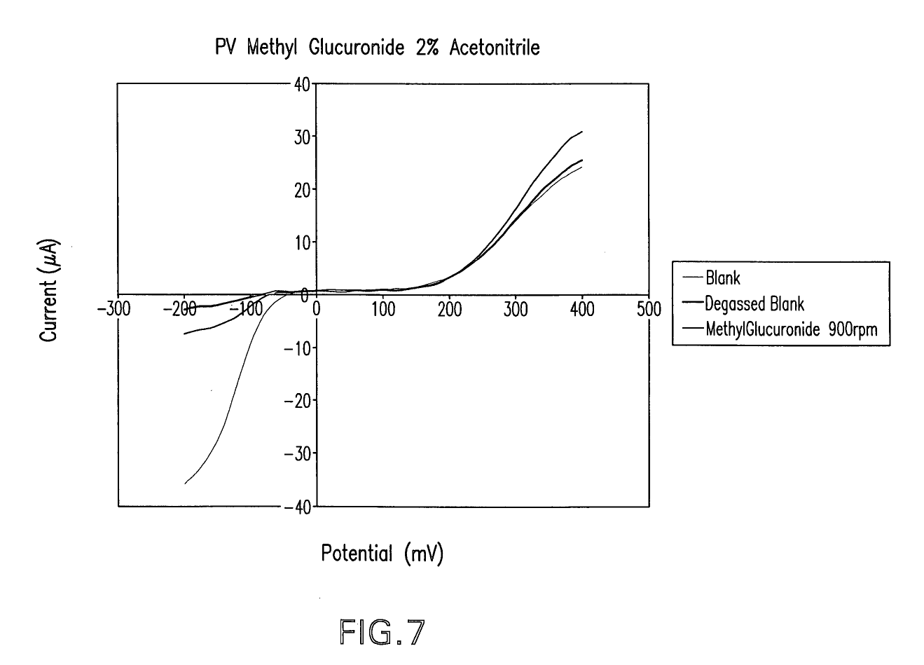

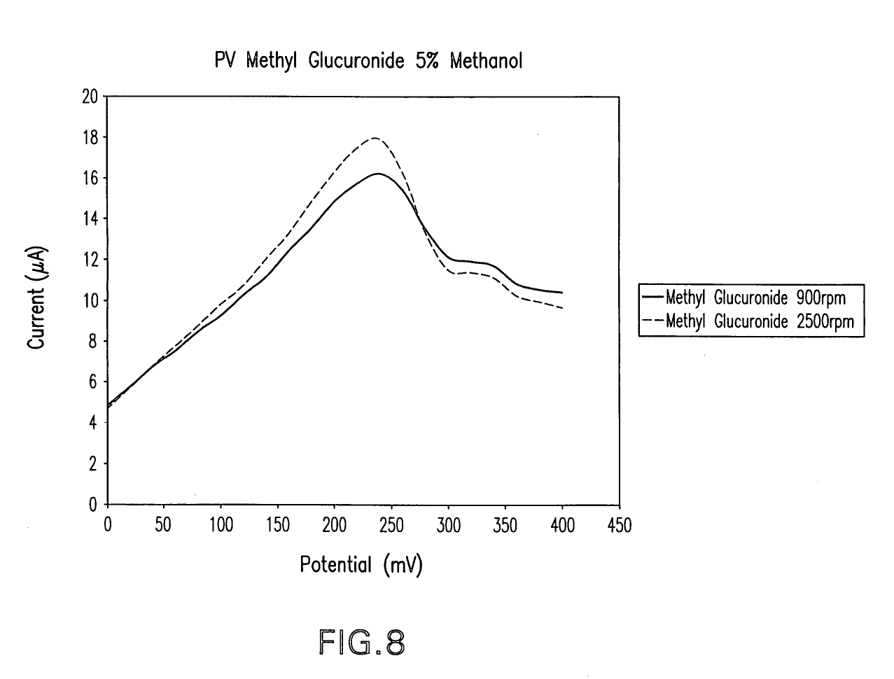

In the presence of 200 mM NaOH only, the detection is not suppressed (FIG. 4). As shown in FIG. 5, the organic interferes with detection of glucuronic acid. A PV of methyl glucuronide generated with only 200 mM NaOH yielded a good signal at +200 mV (FIG. 6). FIG. 6 shows that +200 mV is the optimal potential for detection of the glucuronides. FIG. 7 shows that with the addition of 2% acetonitrile the signal of the compound at +200 mV was suppressed by the presence of acetonitrile in the mobile phase. A PV graph of methyl glucuronide in 5% methanol was generated (FIG. 8) showing a noisy plot. Methanol can interfere with detection at the electrode surface and thus acetonitrile was the organic chosen for the mobile phase in this project.

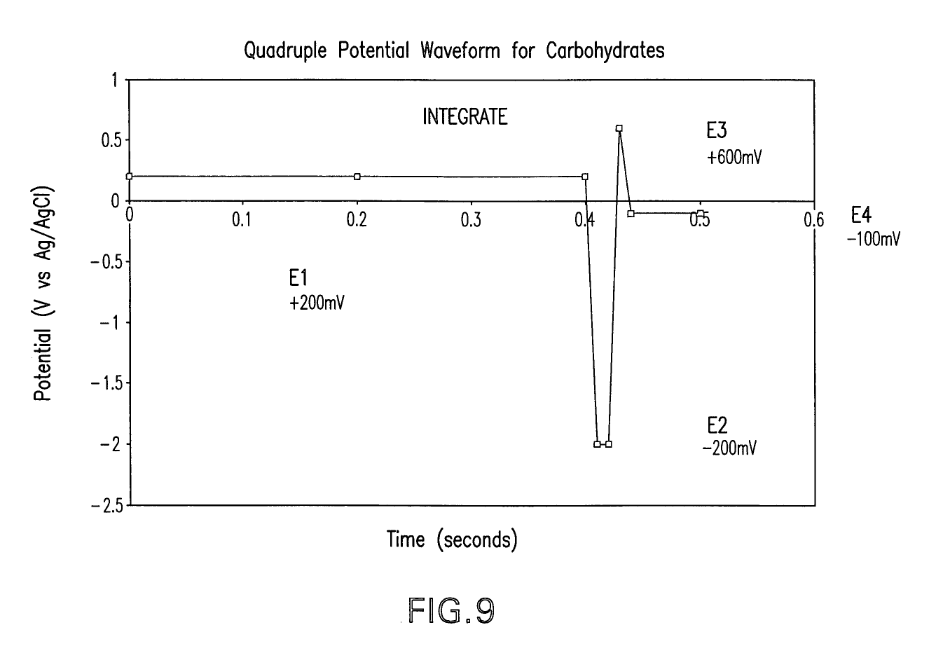

It was determined that 200 mM NaOH resulted in sufficient alkalinity to achieve PED. This required 600 mM NaOH to be delivered via RDM at a flow rate of 0.5 mL/min and mixed with the mobile phase to give a final concentration of 200 mM NaOH. The NaOH was delivered through a mixing tee connected to a mixing coil which bridges the mixing tee and detector. A knitted reaction coil with a weaving pattern achieves good mixing and reduces band-broadening. The nature of the mixing coil is such that backpressure is minimized and coils can be made with Teflon tubing. An exemplary PED waveform that can be used for the detection of glucuronides (for example, alcohol glucuronides) is shown in FIG. 9. Table I lists the values of the optimized waveform.

TABLE I
Optimized PED waveform parameters.

| Potential (mV) vs. | | |
|---|---|---|
| Ag/AgCl | Time (ms) | Integration Interval |
| 200 | 0 | |
| 200 | 200 | begin |
| 200 | 400 | end |
| −1500 | 410 | |
| −1500 | 420 | |
| 600 | 430 | |
| −100 | 440 | |
| −100 | 500 | |

Validation

LC-PED

Alkylglucuronides were readily separated by a reversed-phase mechanism with a mixture of aqueous and organic solvents as the mobile phase. Acetonitrile was chosen because of its compatibility with PED. As expected, the capacity factor (k′) decreases logarithmically as the percentage of acetonitrile in the mobile phase is increased. Due to EtG's high polarity, an organic modifier concentration of 2% acetonitrile was chosen to maximize retention while meeting the minimum organic modifier content required for column stability. In order to decrease the hydrophilicity of EtG, one percent acetic acid was added to the mobile phase to maintain a pH at which EtG is fully protonated. All separations were performed using a mobile phase of 1% acetic acid/ACN, 98/2 v/v. This selection resulted in the baseline resolution of EtG from all interferents from post-mortem urine sample.

Post-column addition of NaOH reagent provides the supporting electrolyte for electrochemical detection and also provides the highly alkaline conditions required for electrocatalytic detection of carbohydrates at noble metal electrodes, and, if used, often ameliorate the effects of any mobile phase buffers or gradients. In an effort to reduce peak dilution, the post-column reagent was made as high in concentration (600 mM NaOH) as allowed by the post-column delivery apparatus, and NaOH was added at a reduced rate (0.6 mL·min-1) relative to the flow rate of the analytical separation (1.0 mL·min-1). In addition to using a zero-dead-volume mixing-tee, the length (i.e., volume) of the mixing coil was minimized to reduce band-broadening effects.

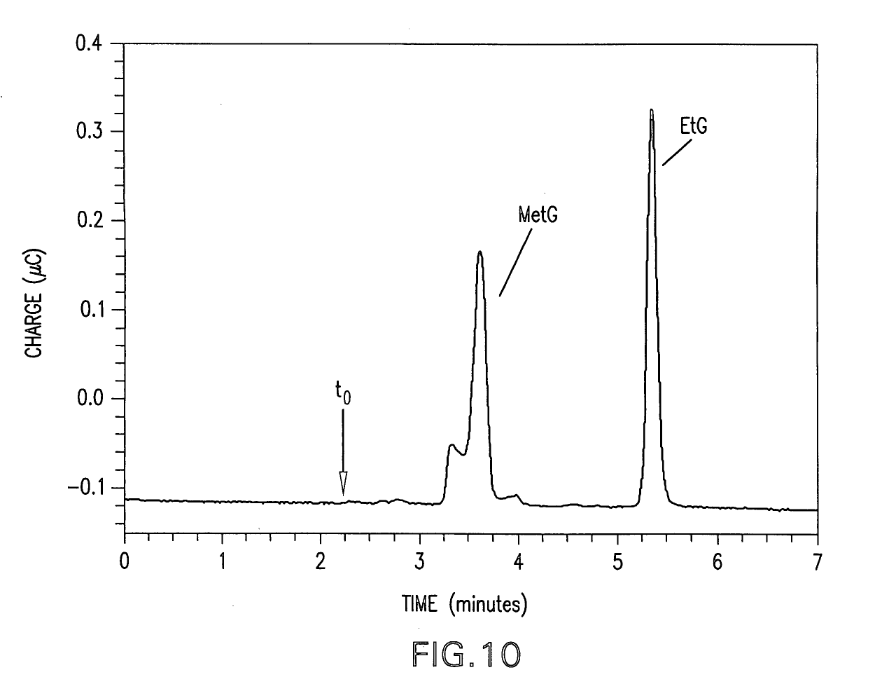

FIG. 10 shows a chromatogram of (a) MetG and (b) EtG under the chromatographic conditions described above. The peaks on either side of MetG are impurities in the MetG standard. Isolated injections of EtG showed no additional peaks. Table II lists the analytical figures of merit for PED using the optimized waveform. All quantitative measurements were based on the peak height measurement of the analytical signal. Regression analysis showed that the PED response was linear over the concentrations (i.e., 200 ppm to the limits of detection (LOD)) tested in water, and LOD for MetG and EtG in water were 0.1 μg/mL and 0.7 μg/mL, respectively. In urine, a linear response was found over the concentration tested, and the detection limit for EtG was determined to be 0.2 μg/mL. Repeatability, or the percent relative standard deviation (% RSD), of repetitive injections at the limit of quantitation (S/N=10) in water and urine were 1.7 and 4.1, respectively.

TABLE II
Quantitative parameters of glucuronides by PED.

| | LOD* | Linear Range | | | LOQ* | % RSD |
|---|---|---|---|---|---|---|
| | (μg/mL, | μC = a(pmol) + b | | | (μg/mL, | (n = 6) |
| Compound | pmol) | a | b | r² | pmol) | 10 S/N |

Cell A - PED cell

| | | | | | | |
|---|---|---|---|---|---|---|
| EtG | 0.1, 20 | 0.041 | 0.016 | 0.99970 | 0.4, 90 | 1.7 |
| MetG | 0.7, 200 | 0.022 | 0.025 | 0.99967 | 2.4, 541 | 4.8 |
| EtG (urine) | 0.2, 5 | 0.093 | 0.0383 | 0.99917 | 0.78, 176 | 4.1 |

Cell B - enhanced electrochemical cell

| | | | | | | |
|---|---|---|---|---|---|---|
| EtG | 0.03, 7 | 0.027 | 0.0022 | 0.99939 | 0.1, 22 | 0.53** |
| MetG | 0.08, 20 | 0.015 | 0.0013 | 0.99903 | 0.3, 57 | 0.79** |

*Limits of detection were determined at 3 times S/N ratio from concentrations within 10 times the LOD.
**% RSD determined at S/N = 100.

Using an enhanced electrochemical cell, the limits of detection (LOD) for EtG and MetG in water were determined to be about 0.03 μg/mL and about 0.08 μg/mL, respectively. The lower limits of detection are matched by a corresponding reduction in the limit of linearity to 10 ppm. Since quantitation is performed at ˜1 ppm, repetitive injections at a S/N=100 resulted in % RSDs less than 1.0 for both EtG and MetG. The low detection limits are particularly notable in light of the fact that alkylglucuronides are not UV active, and absorbance-based methods have little analytical utility.

Three blind samples were analyzed in triplicate by LC-PED using the method described above. Results were determined to be greater then 98% of spiked concentration for all samples with no significant difference between the results and the true value determined at the 95% confidence level, as shown in Table III.

TABLE III
Summary of blind study results.

| | | True Value | Found | |
|---|---|---|---|---|
| Sample # | Sample | (μg/mL) | (μg/mL) | % Recovery |
| 1 | water | 0.80 | 0.79 ± 0.01 | 98.8 ± 1.2 |
| 2 | water | 1.50 | 1.47 ± 0.03 | 98.0 ± 2.0 |
| 3 | water | 2.70 | 2.66 ± 0.04 | 98.5 ± 1.5 |
| 4 | urine | 51.0 | 51.0 ± 0.02 | 100 ± 0.04 |
| 5 | urine | 102 | 102 ± 0.10 | 100 ± 0.10 |
| 6 | urine | 153 | 153 ± 0.05 | 100 ± 0.03 |

LOQ/LOD

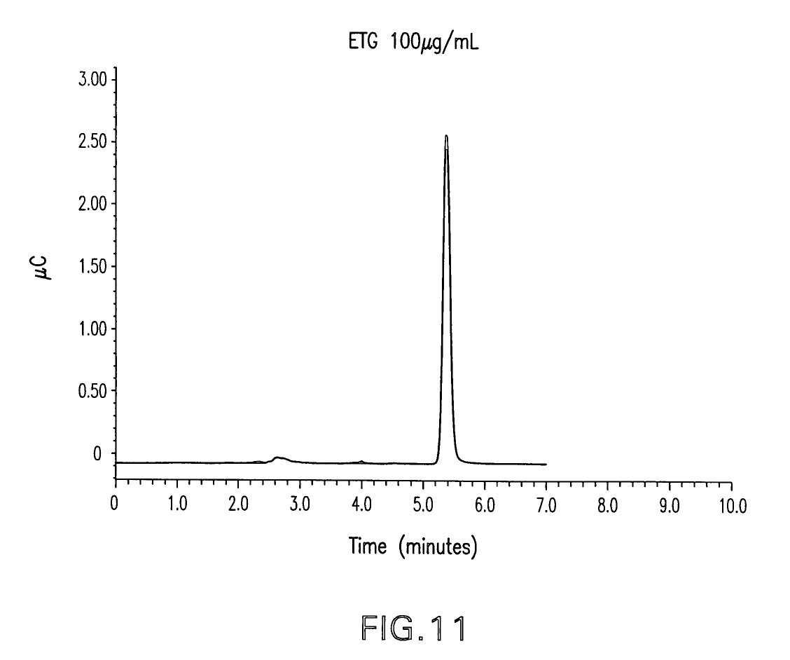

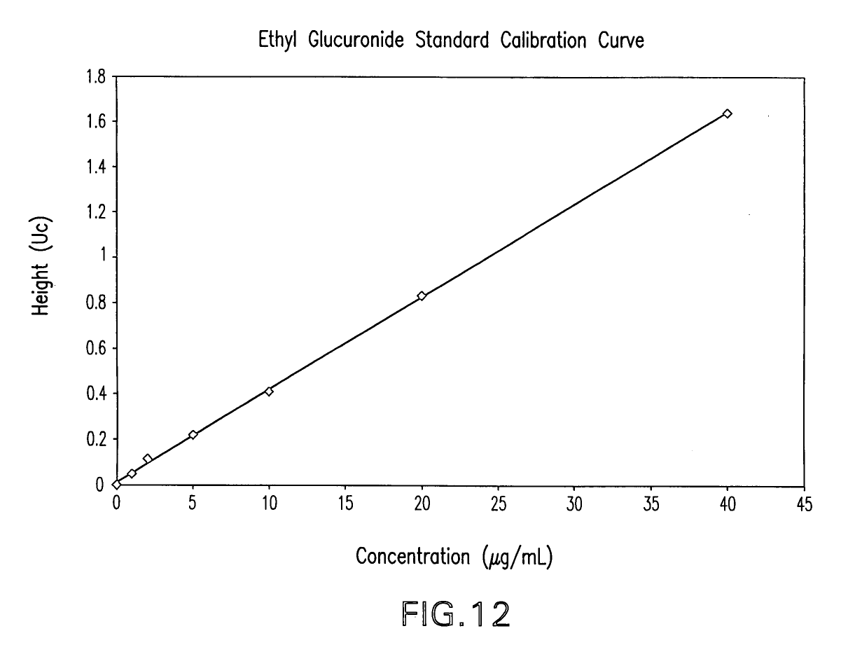

In order to detect EtG the mobile phase described above was used. EtG was found to have a retention time of 5.3 minutes with LOQ and LOD values of about 0.4 and about 0.1 ug/mL respectively (Table IV). FIG. 11 is a representative chromatogram of 100 ug/mL EtG water standard. FIG. 12 shows the calibration curve for water spiked (unextracted) with EtG at different concentrations. A linear response is shown for the analyte in water. An internal standard was used to compensate for errors in the extraction process. Methyl glucuronide was chosen as it has similar physical and chemical properties to ethyl glucuronide and does not co-elute with EtG. The LOQ and LOD values for MetG were determined to be about 2.4 and about 0.7 ppm (Table IV).

TABLE IV
Analytical Figures of Merit for the glucuronides studied.

| Glucuronide | Ethyl | Methyl |
|---|---|---|
| LOQ | 0.4 | 2.4 |
| LOD | 0.1 | 0.7 |
| R² | 0.9997 | 0.9997 |
| A | 0.041 | 0.022 |
| 13 | 0.016 | 0.025 |
| % RSD | 1.7 | 4.8 |
| Height | | |
| % RSD Area | 2.1 | 4.3 |

Upper Limit of Linearity

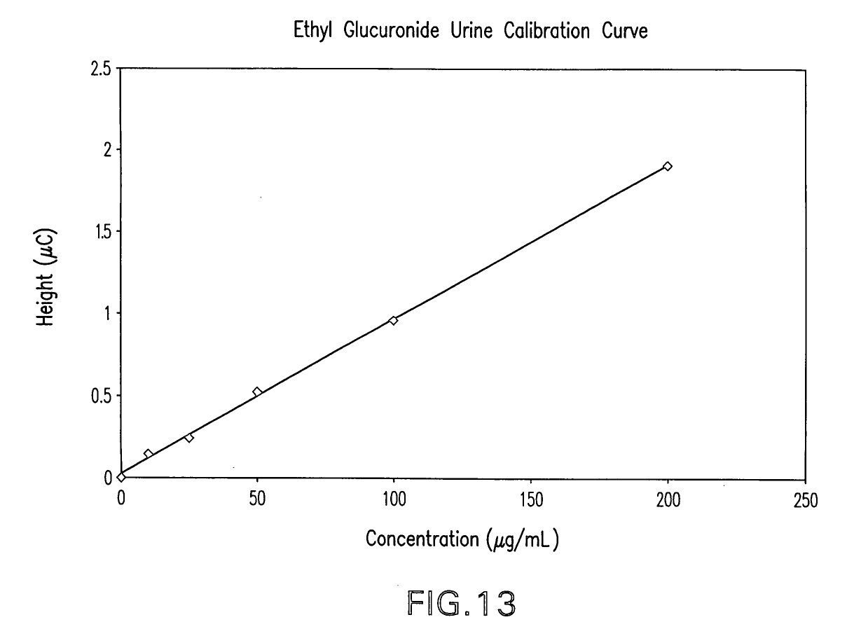

A calibration curve of EtG spiked into urine demonstrates linearity up to 200 ug/mL (FIG. 13). Above 200 ug/mL, it was not possible to see an increase in signal, indicating the limit of linearity. In general, EtG levels in postmortem samples that range from about 3-700 ug/mL.

Solid Phase Extraction

Fractionation of a urine sample to isolate EtG prior to injection was accomplished using a SPE procedure. As noted above, the urine samples (1.0 mL) were pretreated with 100 μL of 3 M HCl and 3 mL acetonitrile to protonate the glucuronides and precipitate proteins, respectively. The 4.2 mL of pretreated specimen was loaded on a conditioned aminopropyl cartridge with a 500 mg (3 cc) sorbent bed, which was selected to maximize recovery. After the cartridge was air-dried under vacuum for 10 minutes to remove any residual water, the glucuronides were eluted with 2% ammonia in methanol. The original procedure called for the use of 2% ammonia in water, but subsequent dry down in the Speed Vac took an excessive amount of time (>30 min). Therefore, the elution solvent was switched to 2% ammonia in methanol as it dries down much faster than water (˜10 min). No loss in recovery was observed. In addition, a two step elution with 2 mL elution solvent per step resulted in higher recoveries and was incorporated in the final extraction procedure. As noted, each sample was reconstituted with 1.0 mL of water and further diluted, if necessary, to bring the EtG level within the linear range of quantitation.

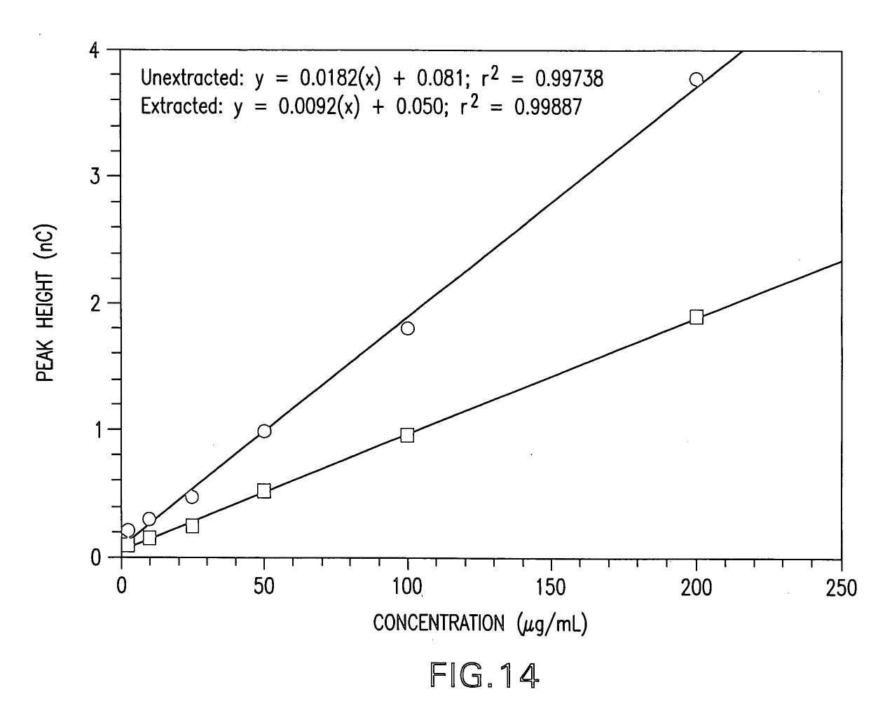

FIG. 14 shows the response of EtG standard solutions (◯) before and (□) after the SPE procedure. Each point represents the average of three individual extractions. By ratioing the slopes of the lines, the average recovery was determined to be 51% with 2.0% RSD. The high linearity of the plots denotes that the recovery of EtG is consistent over the range of concentrations tested. By testing the extraction solutions at every step, it was determined that the majority of the loss of analyte is inadequate retention during the loading process.

Recovery

The SPE recoveries of EtG were determined and are listed in Table V. Recoveries were calculated by comparing the height of the analyte peak measured in the extracted standard to the height of the analyte peak measured directly in unextracted standard containing a known amount of the analyte. The recoveries were approximately 50% and found to be very reproducible over a range of concentrations. There are several reasons for low recoveries in SPE methods. However, 50% consistent recovery is acceptable. The key is that the recovery is reproducible. Low recovery could be due to incomplete retention, loss during washing or incomplete desorption. Incomplete retention could be due to tight protein binding of the analyte, sometimes encountered with urine. The drug bound to a protein of greater than 15,000 daltons, results in a complex too large to retain on the sorbent bed. Instead the protein would pass through the sorbent unretained and would drag the bound analyte with it. There is also evidence that amine-containing species disrupt secondary interactions between analyte and sorbent. This reduces retention and makes elution problematic. The addition of a small amount of ammonium hydroxide to the eluting solvent breaks up protein binding and weakens the secondary interactions allowing optimal elution of the analyte. The other possibility is incomplete desorption due to very strong interactions between the analyte and sorbent. However, interactions involving aminopropyl columns are low to moderately strong. In the present example, the analyte was not washed out during the acetonitrile washing step. This was determined by injecting a sample of the collected wash, which showed no EtG peak. Therefore, prevention of higher recoveries was probably due to incomplete retention.

TABLE V
Solid phase extraction recoveries for EtG. Extractions were done
using 3 cc Sep-pak vac aminopropyl cartridges with 500 mg sorbent.

| EtG | | | |
|---|---|---|---|
| Levels | Extracted Std | Unextracted Std | % |
| (ug/mL) | Peak Height | Peak Height | Recovery |
| 200 | 1.906719 | 3.768321 | 50.6 |
| 100 | 0.959364 | 1.802515 | 53.2 |
| 50 | 0.524547 | 0.987541 | 53.1 |
| 25 | 0.242023 | 0.46521 | 52 |
| 10 | 0.148895 | 0.294828 | 50.5 |
| 2 | 0.095343 | 0.198593 | 48.1 |

Precision

As listed in Table IV the EtG chromatograms show good reproducibility as reflected in % RSD values of 1.74 for peak height and 2.15 for peak area. In addition, chromatograms of MetG are reproducible shown in Table IV by the % RSD values of 4.8 and 4.3. For each extracted case, samples were run in triplicate on the HPLC. The within run % RSD values are reported and ranged from 0.1%-4.8% (Table VI).

TABLE VI
Ethyl glucuronide levels are reported along with the alcohol
concentration in the blood, urine and vitreous humor. The cases
were run in triplicate on the HPLC and reproducibility is
demonstrated by the % RSD values listed.

| | | | | | Vitreous |
|---|---|---|---|---|---|
| CASE | % RSD | BAC | EtG Levels | UAC | Humor |
| # | (n = 3) | (ug/mL) | (ug/mL) | (ug/mL) | (ug/mL) |
| 2972 | 0.6 | 1.5 | 800 | 2.7 | 2.3 |
| 2995 | 1.5 | 1.1 | 550 | 1.4 | 1.1 |
| 3018 | 4.4 | 1.9 | 171 | 2.6 | 2.2 |
| 3023 | 4.2 | 2.5 | 154 | 3.9 | 2.8 |
| 3036 | 2.2 | 0.8 | 800 | 1.4 | 1.1 |
| 3037 | 2.8 | 0.9 | 513 | 0.9 | 1.2 |
| 3048 | 3.1 | 3.7 | 109 | 2.2 | 1.8 |
| 3064 | 0.6 | 0.4 | 47 | 0.5 | 0.8 |
| 3598 | 2.5 | 2.5 | 1150 | 2.5 | 3.1 |
| 3594 | 1.7 | 2.4 | 1176 | 2.6 | 2.2 |
| 3592 | 4.8 | 0.5 | 111 | 0.5 | 0 |
| 3570 | 1.4 | 2.4 | 575 | 2.8 | 2.6 |
| 3555 | 0.3 | 2.2 | 63 | 1.6 | 1.7 |
| 3554 | 2.3 | 3.5 | 1371 | 3.1 | 3.1 |
| 3541 | 2.4 | 0.8 | 127 | 1.1 | 0.9 |
| 3538 | 0.6 | 0.4 | 553 | 1.7 | 0.7 |
| 3533 | 1.4 | 0.7 | 61 | 0.9 | 0.6 |
| 3527 | 4.2 | 0.7 | 636 | 1.9 | 0.9 |
| 3523 | 4.9 | 2.1 | 114 | 2.9 | 2.4 |
| 3519 | 2.8 | 1.1 | 550 | 2 | 1.8 |
| 3513 | 0.1 | 2.1 | 765 | 2.3 | 2.2 |

Postmortem Urine Samples

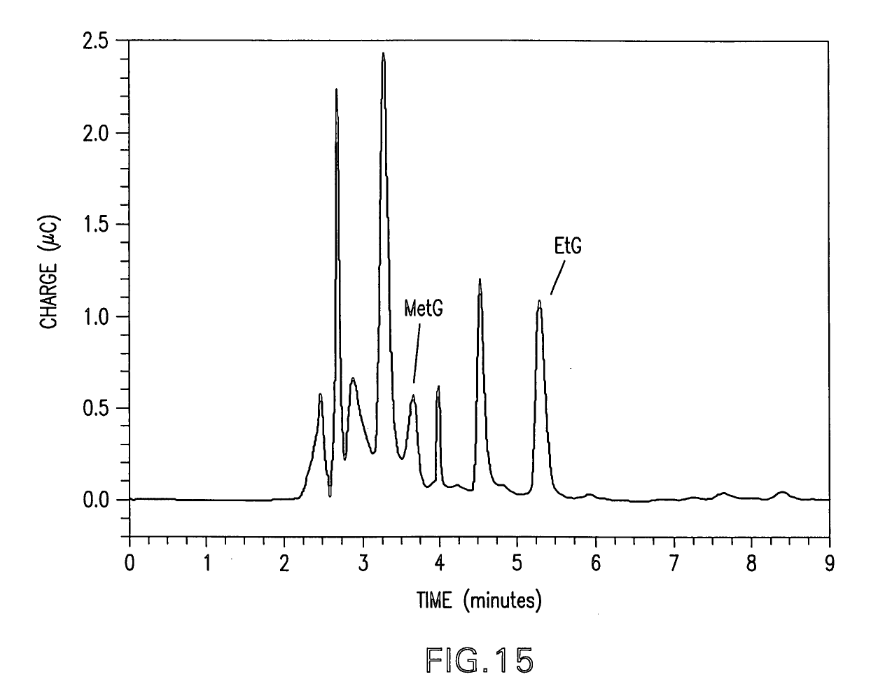

FIG. 15 is a typical chromatogram of a postmortem case identifying the peaks for EtG and MetG, which serves as an internal standard. The target range of EtG in post-mortem urine samples is expected to be in the range of 5 to 700 μm/mL, which is easily achieved with LC-PED by sample dilution. EtG is clearly baseline resolved from potential interferents. For all tested samples, the unspiked injection showed no endogenous MetG, and quantitation by peak height measurement of MetG spiked into blank urine samples were highly reproducible (<2% RSD). Quantitation using MetG as an internal standard produced no bias in the quantitation of EtG. Other internal standards that could be completely resolved and elute after the EtG peak and interfering matrix components, such as propyl or t-butyl glucuronide, were not commercially available.

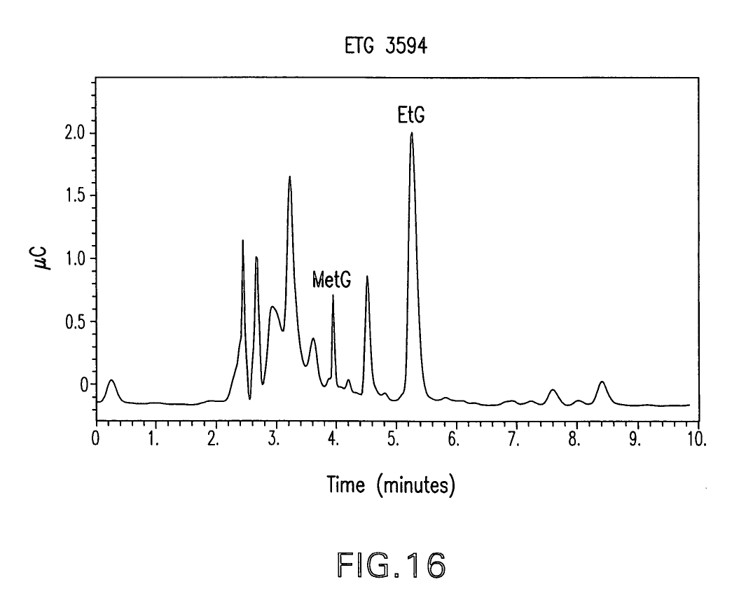

FIG. 16 is a chromatogram of postmortem case #3594 with MetG spiked into the sample serving as the internal standard. It should be noted that the MetG peak is partially unresolved and lies within interfering components of the matrix. However, peak height measurements were useful in determining EtG concentrations in case samples. In addition, other internal standards that could be completely resolved and elute after the EtG peak and interfering matrix components such as propyl or t-butyl glucuronide were not commercially available at the time the experiment was performed. Therefore, it was deemed that MetG would suffice as an internal standard.

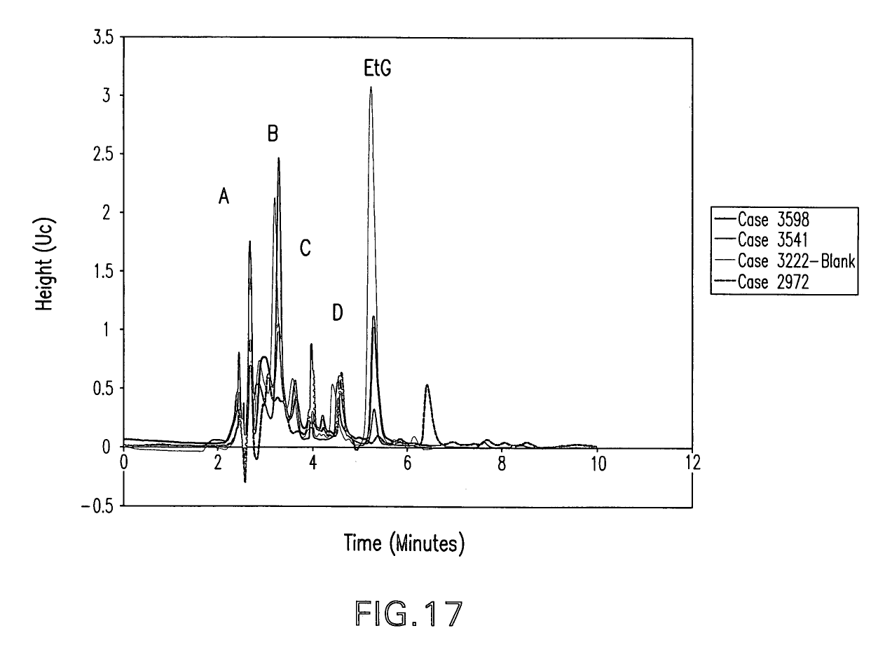

To determine if EtG correlated with UAC and BAC better than other unknown peaks in a sample, 4 unknown peaks were chosen. These peaks were present in all urine cases and corresponded to the following retention times; 2.68, 3.2, 3.97 and 4.42 mins. These retention times shifted slightly from case to case but remained approximately the same. FIG. 17 shows 3 postmortem case chromatograms overlapped with different levels of EtG. A case that contained no alcohol is included as the blank or control (FIG. 17). All blank cases ran did not contain EtG. These chromatograms clearly show the four unknown peaks that are consistently present in all case samples labeled A-D. In Table VII the strength of the relationships between EtG and the other unknown peaks with BAC and UAC is listed. Relative to the other peaks EtG correlates the best with both BAC and UAC.

TABLE VII
A comparison of correlation coefficients of EtG values and 4
unknown peaks that appeared in all case samples versus blood and
urine alcohol concentrations.

| | BAC- R | UAC- R |
|---|---|---|
| | value | value |
| EtG | 0.30 | 0.37 |
| Unknown | 0.21 | 0.23 |
| A | | |
| Unknown | 0.11 | 0.15 |
| B | | |
| Unknown | 0.33 | 0.26 |
| C | | |
| Unknown | 0.07 | 0.017 |
| D | | |

Table VIII lists the EtG levels found in each of the 29 post-mortem samples tested. Each of the urine samples were run in triplicate and the reported % RSD values ranged from 0.1%-4.9%. Furthermore, the vitreous humor, blood, and urine ethanol concentrations from each of the cases are also listed. Of the 29 total samples, eight control cases from individuals who did not consume alcohol prior to death were assayed. In all control cases, no EtG was found.

TABLE VIII
EtG levels are reported along with the alcohol concentration in the
vitreous humor, blood, and urine.

| | Vitreous | | | | % |
|---|---|---|---|---|---|
| | Humor | BAC | UAC | EtG | RSD |
| CASE # | (μg/mL) | (μg/mL) | (μg/mL) | (μg/mL) | (n = 3) |
| 2972 | 2300 | 1500 | 2700 | 800 | 0.6 |
| 2995 | 1100 | 1100 | 1400 | 550 | 1.5 |
| 3018 | 2200 | 1900 | 2600 | 171 | 4.4 |
| 3023 | 2800 | 2500 | 3900 | 154 | 4.2 |
| 3036 | 1100 | 800 | 1400 | 800 | 2.2 |
| 3037 | 1200 | 900 | 900 | 513 | 2.8 |
| 3048 | 1800 | 3700 | 2200 | 109 | 3.1 |
| 3064 | 800 | 400 | 500 | 47 | 0.6 |
| 3598 | 3100 | 2500 | 2500 | 1150 | 2.5 |
| 3594 | 2200 | 2400 | 2600 | 1176 | 1.7 |
| 3592 | 0 | 500 | 500 | 111 | 4.8 |
| 3570 | 2600 | 2400 | 2800 | 575 | 1.4 |
| 3555 | 1700 | 2200 | 1600 | 63 | 0.3 |
| 3554 | 3100 | 3500 | 3100 | 1371 | 2.3 |
| 3541 | 900 | 800 | 1100 | 127 | 2.4 |
| 3538 | 700 | 400 | 1700 | 553 | 0.6 |
| 3533 | 600 | 700 | 900 | 61 | 1.4 |
| 3527 | 900 | 700 | 1900 | 636 | 4.2 |
| 3523 | 2400 | 2100 | 2900 | 114 | 4.9 |
| 3519 | 1800 | 1100 | 2000 | 550 | 2.8 |
| 3513 | 2200 | 2100 | 2300 | 765 | 0.1 |

Control Cases

| | | | | | |
|---|---|---|---|---|---|
| 3143 | 0 | 0 | 0 | ND | — |
| 3145 | 0 | 0 | 0 | ND | — |
| 3178 | 0 | 0 | 0 | ND | — |
| 3182 | 0 | 0 | 0 | ND | — |
| 3208 | 0 | 0 | 0 | ND | — |
| 3222 | 0 | 0 | 0 | ND | — |
| 3226 | 0 | 0 | 0 | ND | — |
| 3239 | 0 | 0 | 0 | ND | — |

ND denotes not detected.

Correlations

In one embodiment, the present invention provides methods to determine whether or not a person had consumed alcohol prior to death. The presence of EtG in urine is expected to be found at various levels for drinkers, depending on the amount of alcohol consumed, and not found for teetotalers. Vitreous humor alcohol (VHAC), blood alcohol (BAC), and urine alcohol concentrations (UAC) for all cases were obtained from the Office of the Chief Medical Examiner, MD. These results confirm the expectations, in that, the VHAC, BAC, and UAC were 0 in all cases, which correlates well with no EtG found in any of the control cases. For all specimens that had measurable VHAC, BAC, and UAC, EtG was also found at various levels.

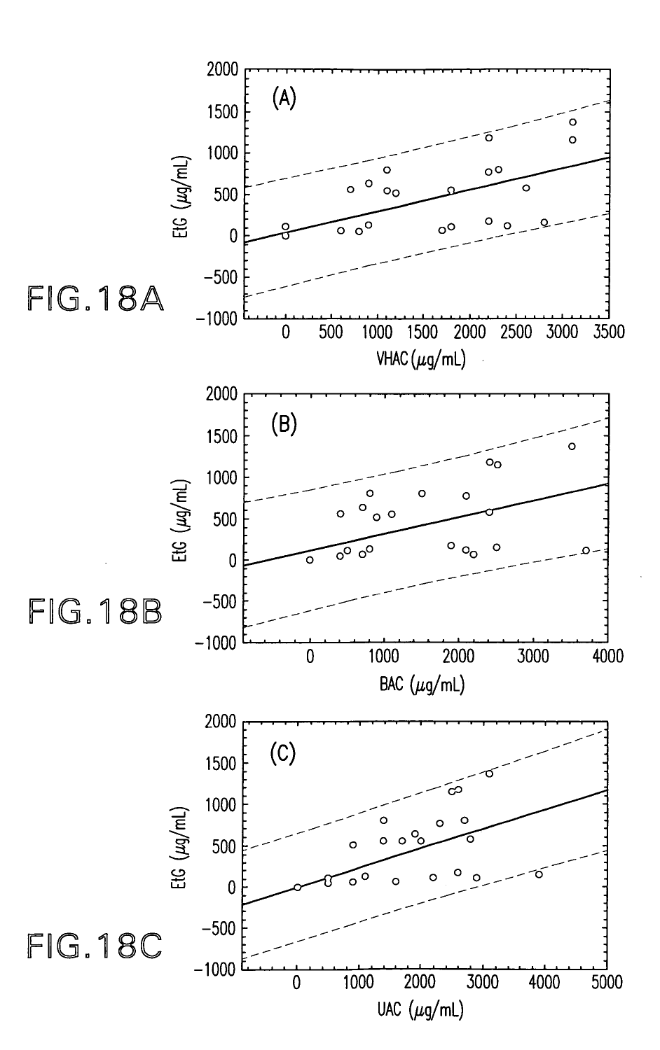

EtG concentrations were plotted against VHAC (FIG. 18A), BAC (FIG. 18B), and UAC (FIG. 18C). There was a positive correlation found between EtG levels and VHAC (r=0.6718), BAC (r=0.5499), and UAC (r=0.6177). It is difficult to compare the amount of EtG and alcohol measured in the urine due to the fact that the concentration of EtG can be lowered considerably by increased water intake which increases urine flow rate. However, alcohol concentration in urine is not affected by drinking large amounts of water because it is excreted by the kidney by passive diffusion. Other reasons may include inter-individual variations and differences in the time-lag between individuals EtG excretion profiles. A strong quantitative correlation between EtG urine and BAC is not expected due to a myriad of reasons, such as alcohol is cleared very quickly from the blood and there are individual differences in alcohol distribution and elimination; genetic polymorphisms for the presence and activity of UDP-glucuronosyltransferase (UGT); and the distribution of EtG into systemic circulation is delayed compared to blood ethanol distribution. However, even without correction for dilution by measuring creatinine, a moderate correlation exists between EtG and the certified measurements with the strongest correlation between EtG and VHAC.

As to the determination of alcohol consumption prior to death, the presence of EtG in urine can be suggested as a first-choice test. Also, as EtG has a longer half-life than ethanol an accumulation in urine of the compound would be expected. This allows for recent alcohol consumption to be disclosed after ethanol is no longer measurable in body fluids.

### CONCLUSION

LC-PED is a sensitive, selective, and direct method for the determination of glucuronide analogues (e.g., EtG) in urine. Derivatization is not necessary, and the technique is of a much lower cost than LC-MS. The SPE method used was highly reproducible and only requires 1 mL of sample. This method shows promise as a tool for the determination of alcohol consumption, and it can be used to distinguish between alcohol ingested prior to death and alcohol formed postmortem during decomposition.

Detection of Additional Glucuronides

The method disclosed herein are applicable as detection/screening techniques for any glucuronides, including those disclosed throughout, such as codeine, lorazepam and acetaminophen glucuronide. To that end, ethyl glucuronide has served as a model compound for the development of a general glucuronide detection/screening technique. Thus, an entire glucuronide profile can be produced that indicates all of the drugs/compounds that a person has consumed in a single analysis.

Another exemplary glucuronide metabolite is morphine glucuronide. Morphine is mainly metabolized by conjugation with glucuronic acid via UDP-glucuronyltransferase forming morphine-3-glucuronide (M3G) and morphine-6-glucuronide (M6G).

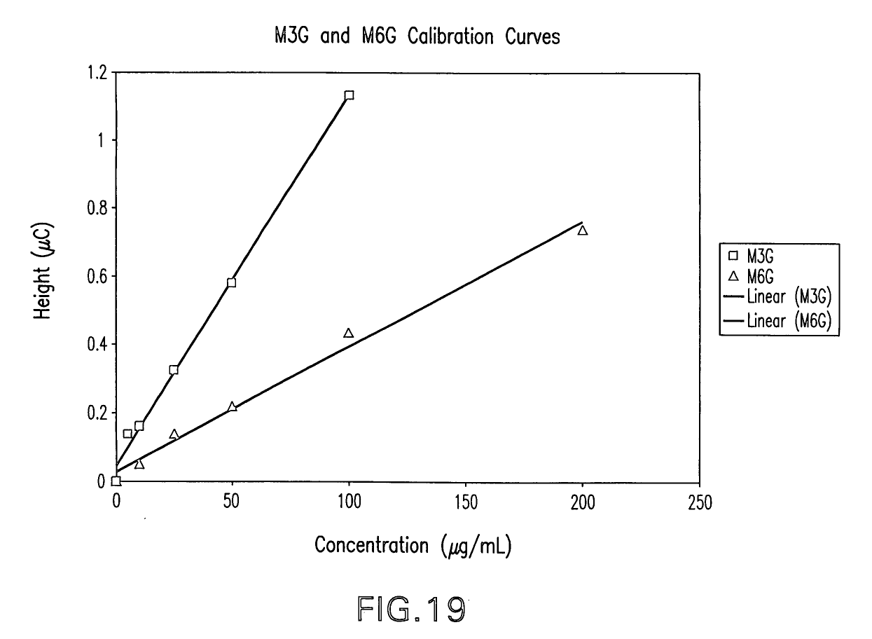

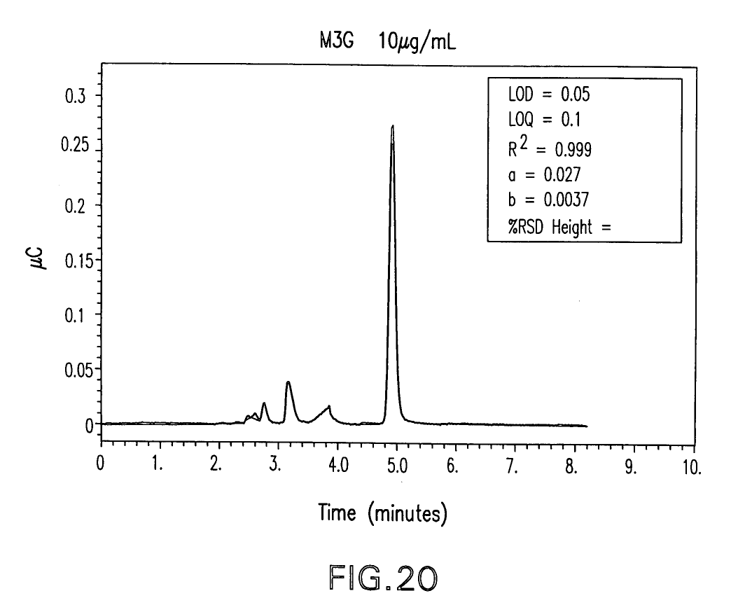

In order to run the morphine metabolites the composition of the mobile phase had to be changed from that used for EtG. To retain M3G and provide good peak shape, 3% ACN/water with 1% acetic acid was used as the mobile phase. The LOQ and LOD values of M3G are 0.1 and 0.05 respectively. For the separation of M6G the mobile phase consisted of 8% ACN/water with 1% acetic acid. Logically both metabolites were injected together since, in real postmortem cases, both would be present after ingestion of morphine. FIG. 19 shows the standard calibration curves of the morphine glucuronides. As illustrated in FIGS. 20 & 21, which are representative chromatograms of M3G and M6G, their retention times are 5.18 and 4.17 respectively.

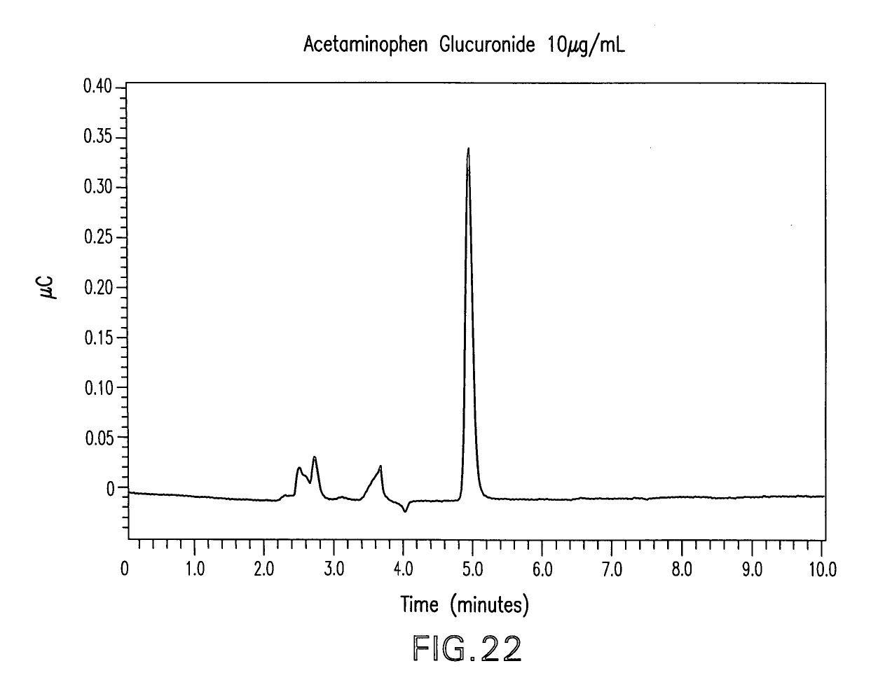

It should be noted that acetaminophen glucuronide (FIG. 22) was also run and successfully detected using these methods. This illustrates the broad scope of this technique which can be extended to the detection of all glucuronides in a human urine sample. Finally, this technique is rugged, inexpensive, provides good reproducibility, and is easy to use. It provides good quantitative and qualitative information and does not require derivatization procedures.

### Example 2

### Glucuronide Detection Using t-Butanol

t-Butanol was substituted for acetonitrile of the mobile phase described above consisting resulting a phase comprising about 1% acetic acid/water:t-butanol (98:2). The result of this replacement was a 5-fold increase in signal of the analyte of interest. Solid-phase extraction recoveries for EtG were 50% using aminopropyl columns. Due to the fact that the internal standard peak corresponding to methyl glucuronide (MetG) lay within the interference of the matrix, propyl glucuronide, which is baseline resolved and elutes out of the interference region, was used as the internal standard. The LOQ and LOD for EtG were improved to 0.02 and 0.007 ug/mL, from previous values of 0.4 and 0.1 ug/mL, respectively. A bonded-phase silica column comprising a C18 functional group bonded to its surface using a sulfonamide group coupled to an ether linkage (DIONEX® PA column, DIONEX®, Sunnyvale, Calif.) was used as it provides better peak shape and resolution. Blind studies in urine showed no significant difference between the results and the true value determined at the 95% confidence level, for all samples.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A method for detecting one or more glucuronide metabolites in a liquid sample, comprising:
(a) adding an organic solvent to the liquid sample to form a mixture;
(b) passing the mixture through one or more analytical chromatographic columns, thereby separating the one or more glucuronide metabolites and producing an eluate;
(c) adding NaOH to the eluate; and
(d) detecting one or more glucuronide components of the separated glucuronide metabolites with an electrochemical detector.

2. The method of claim 1, further comprising:
passing the mixture in (a) through one or more pre-concentration chromatographic columns, thereby retaining the one or more glucuronide metabolites on the one or more pre-concentration chromatographic columns and concentrating the one or more glucuronide metabolites; and
delivering a solvent to the one or more pre-concentration chromatographic columns, thereby eluting the one or more glucuronide metabolites from the one or more pre-concentration chromatographic columns to form a mixture to be passed in (b).

3. The method of claim 1, wherein said method is used to detect one or more glucuronide components produced by glucuronidation of an alcohol, morphine, cannabinoid, an androgen, acetaminophen, codeine, buprenorphine or tramadol.

4. The method of claim 3, wherein said method is used to analyze a mixture of glucuronide components produced by glucuronidation of an alcohol, morphine, cannabinoid, an androgen, acetaminophen, codeine, buprenorphine or tramadol.

5. The method of claim 1, wherein said detecting in (d) comprises providing a potential time waveform to an electrode of the electrochemical detector.

6. The method of claim 1, wherein the liquid sample is a physiological liquid.

7. The method of claim 6, wherein the physiological liquid is urine.

8. The method of claim 1, wherein the organic solvent is t-butyl alcohol.

9. A method for detecting one or more alcohol glucuronides of a liquid sample, comprising:
(a) adding an organic solvent to the liquid sample to form a mixture;
(b) passing the mixture through one or more analytical chromatographic columns, thereby separating the one or more alcohol glucuronides;
(c) adding NaOH to the separated one or more alcohol glucuronide; and
(d) detecting one or more glucuronide components of the separated alcohol glucuronides in an electrochemical detector.

10. The method of claim 9, further comprising, prior to said passing in (b):
passing the mixture in (a) through one or more pre-concentration chromatographic columns, thereby retaining the one or more alcohol glucuronides on the one or more pre-concentration chromatographic columns and concentrating the one or more glucuronide metabolites; and
delivering a solvent to the one or more pre-concentration chromatographic columns, thereby eluting the one or more alcohol glucuronides from the one or more pre-concentration chromatographic columns to form a mixture to be passed in (b).

11. The method of claim 9, wherein the one or more alcohol glucuronides are selected from the group consisting of methyl glucuronide, ethyl glucuronide, butyl glucuronide and propyl glucuronide.

12. The method of claim 9, wherein said detecting in (d) comprises providing a potential time waveform to an electrode of the electrochemical detector.

13. The method of claim 9, wherein at least one of the one or more chromatographic columns is a bonded-phase silica column.

14. The method of claim 9, wherein the organic solvent is t-butyl alcohol.

15. The method of claim 9, wherein the liquid sample is a physiological liquid.

16. The method of claim 15, wherein the physiological liquid is urine.

17. A glucuronide analysis kit comprising:
(a) one or more chromatographic columns;
(b) one or more organic solvents;
(c) one or more glucuronide standards; and
(d) NaOH.

18. The kit of claim 17, wherein at least one of said chromatographic columns is a bonded-phase silica column.

19. The kit of claim 17, wherein at least one of said organic solvents is t-butyl alcohol.

20. The kit of claim 17, wherein at least one of said glucuronide standards is methyl glucuronide.

21. The kit of claim 17, wherein at least one of said chromatographic columns is a bonded-phase silica column, at least one of said organic solvents is t-butyl alcohol and at least one of said glucuronide standards is methyl glucuronide.

22. A method for determining the prior consumption of a drug or alcohol by an animal, comprising:
(a) obtaining a physiologic liquid sample from the animal comprising one or more glucuronide metabolites of the drug or alcohol;
(b) adding an organic solvent to the liquid sample to form a mixture;
(c) passing the mixture through one or more analytical chromatographic columns, thereby separating one or more glucuronide metabolites and producing an eluate;
(d) adding NaOH to the eluate;
(e) detecting one or more glucuronide components of the separated glucuronide metabolites with an electrochemical detector; and
(f) correlating the one or more glucuronides detected with one or more drugs or alcohols consumed by the animal.

23. The method of claim 22, further comprising:
passing the mixture in (b) through one or more pre-concentration chromatographic columns, thereby retaining one or more glucuronide metabolites on the one or more pre-concentration chromatographic columns and concentrating the one or more glucuronide metabolites; and
delivering a solvent to the one or more pre-concentration chromatographic columns, thereby eluting the one or more glucuronide metabolites from the one or more pre-concentration chromatographic columns to form a mixture to be passed in (c).

24. The method of claim 22, wherein said method is used to detect one or more glucuronide components produced by glucuronidation of an alcohol, cocaine, morphine, cannabinoid, methamphetamine, an androgen, acetaminophen, codeine, buprenorphine or tramadol.

25. The method of claim 22, wherein said detecting in (e) comprises providing a potential time waveform to an electrode of the electrochemical detector.

26. The method of claim 22, wherein the physiological liquid is urine.

27. The method of claim 22, wherein the organic solvent is t-butyl alcohol.

28. The method of claim 22, wherein the animal is a human.
